(19) United States
(12) Patent Application Publication
(10) Pub. No.: US 2014/0073637 A1
(43) Pub. Date: Mar. 13, 2014

(54) AMINO-SUBSTITUTED-ALKYLOXY-BENZO[E]PYRIDO[4,3-B]INDOLE DERIVATIVES AS NEW POTENT KINASE INHIBITORS

(71) Applicants: INSTITUT CURIE, PARIS CEDEX 05 (FR); INSERM (INSTITUT NATIONAL DE LA SANTE ET DE LA RECHERCHE MEDICALE), PARIS CEDEX 13 (FR); CENTRE NATIONAL DE LA RECHERCHE SCIENTIFIQUE, PARIS (FR)

(72) Inventors: Chi-Hung N'Guyen, Antony (FR); Annie Molla, Meylan (FR); Ly-Thuy-Tram Le, Grenoble Cedex (FR)

(73) Assignees: INSTITUT CURIE, PARIS CEDEX 05 (FR); INSERM (INSTITUT NATIONAL DE LA SANTE ET DE LA RECHERCHE MEDICALE, PARIS CEDEX 13 (FR); CENTRE NATIONAL DE LA RECHERCHE SCIENTIFIQUE, PARIS (FR)

(21) Appl. No.: 14/119,535

(22) PCT Filed: May 30, 2012
(86) PCT No.: PCT/EP2012/060083

§ 371 Date: Nov. 22, 2013

(30) Foreign Application Priority Data
May 31, 2011 (EP) 11305659.2

(51) Int. Cl. C07D 471/04; A61K 45/06; A61K 31/5377; A61K 31/437

(52) U.S. Cl. CPC C07D 471/04; A61K 31/437; A61K 45/06; A61K 31/5377
USPC 514/232.8; 546/70; 514/285; 544/125

(57) ABSTRACT

The present invention relates to a new class of benzo[e]pyrido-indole, the amino-substituted-alkyloxy-benzo[e]pyrido[4,3-b]indole derivatives, having a particular kinase inhibition profile and useful as a therapeutic agent, in particular an anti-tumoral agent.

### FIELD OF THE INVENTION

The present invention relates to the medical field and in particular to the oncology field.

### BACKGROUND OF THE INVENTION

Inhibitors of kinases represent a real hope in cancer therapies since the encouraging results obtained with imatinib in leukemia. In particular, Aurora kinases are a family of serine/threonine protein kinases that play a key role in mitosis progression. Aurora A is found to be associated first with centrosomes and finally with microtubules, whereas aurora B is a chromosomal passenger protein. Aurora A is required for centrosome duplication, entry into mitosis, formation of bipolar spindle and mitotic checkpoint. Aurora B exhibits typical passenger protein behavior during mitosis. Initially, the kinase associates with centromeres, and as mitosis proceeds, it relocates to the central spindle and the midbody. Aurora B is essential for chromosome condensation, kinetochore functions, spindle checkpoint activation and cytokinesis completion.

Aurora A and B are overexpressed in many cancers, including primary colon and breast cancer. Furthermore, the human Aurora A gene is localized to the 20q13 amplicon, which is associated with a poor prognosis in breast cancer. Xenografts of mouse NIH-3T3 cells overexpressing aurora A give rise to tumors in nude mice, suggesting that aurora A behaves as an oncogene. Under similar conditions, overexpression of aurora B may induce metastasis.

Benzo[e]pyrido-indoles have been identified as interesting mitotic kinase inhibitors (Hoang et al, 2009). These compounds were found to inhibit aurora kinases with a minimal toxicity. It was also shown that those compounds, in particular compounds C1 and C2, inhibit the growth of different cell lines derived from different carcinoma.

Despite an intensive research, two major problems in cancer therapy remain: the induction of tumor resistance to already used drugs and the balance between the toxicity of the treatment and its efficiency. Therefore, new treatments with less toxicity and resistance induction are still required.

### SUMMARY OF THE INVENTION

In the present invention, the inventors identified a new class of benzo[e]pyrido-indole, the amino-substituted-alkyloxy-benzo[e]pyrido[4, 3-1)]indole derivatives. This new class of compounds presents a therapeutic interest, in particular as an antiproliferative drug.

The present invention relates to a compound having the formula (I) or (II)

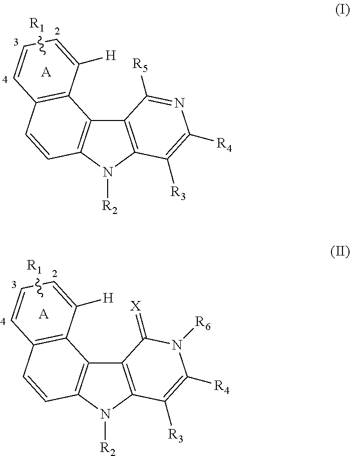

wherein

the benzo cycle A is mono-substituted by R1 in position 2, 3 or 4;

R1 is a radical O—(C₂-C₅)alkyl-NRₐR_{b}, wherein (C₂-C₅)alkyl is a linear or branched alkyl, Rₐ and R_{b}, each independently, are selected from the group consisting of hydrogen, a (C₁-C₄)alkyl optionally substituted by a radical selected from the group consisting of hydroxyl, —NRR′, —OPO(OR)(OR′), and —OC(═O)R, and a (C₃-C₆)cycloalkyl; or NRₐR_{b} may be taken together to form a heterocycle selected from the group consisting of aziridine, azetidine, pyrrolidine, pyrrole, piperidine, piperazine, morpholine, and thiomorpholine, the said heterocycle being optionally substituted by (C₁-C₄)alkyl or hydroxyl radical;

R2 is selected from the group consisting of hydrogen and a (C₁-C₃)alkyl optionally substituted by a radical OH, (C₁-C₃)alkyloxy or —NRR′,

R3 and R4, each independently, are selected from the group consisting of hydrogen, (C₁-C₃)alkyl and aryl; or R3 and R4 may be taken together to form a bivalent radical of formula
—(CH₂)ₙ— wherein n is 3, 4 or 5; or
—CH═CH—CH═CH—, optionally substituted by a (C₁-C₃)alkyloxy;

R5, only present in formula (I), is a (C₁-C₄)alkoxy;

R6, only present in formula (II), is selected from the group consisting of hydrogen and a (C₁-C₃)alkyl, optionally substituted by a radical selected from the group consisting of hydroxyl, —NRR′, —OPO(OR)(OR′), and —OC(═O)R;

X, only present in formula (II), is O or S;

wherein R and R′, identical or different, are selected from the group consisting of hydrogen and a (C₁-C₄)alkyl;

or an isomeric form thereof or a pharmaceutically acceptable salt thereof or a derivative thereof.

Preferably, the compound of formula (I) or (II), more preferably of formula (II), has one or several of the following features:
R1 is a radical O—(C₂-C₄)alkyl-NRₐR_{b}, wherein (C₂-C₄)alkyl is a linear or branched alkyl, Rₐ and R_{b}, each independently, are selected from the group consisting of hydrogen and a (C₁-C₃)alkyl; or NRₐR_{b} may be taken together to form a heterocycle selected from the group consisting of aziridine, azetidine, pyrrolidine, pyrrole, piperidine, piperazine, morpholino and thiomorpholine; and/or
R2 is selected from the group consisting of hydrogen, methyl, ethyl, and —(CH₂)ₙ—N[(C₁-C₂)alkyl]₂ with n being 2 or 3; and/or
R3 and R4, each independently, are selected from the group consisting of hydrogen, methyl, ethyl and phenyl, or may be taken together to form a bivalent radical of formula —CH═CH—CH═CH—; and/or
in formula (I), R5 is a (C₁-C2)alkoxy; and/or
in formula (II), X is an oxygen; and/or
in formula (II), R6 is selected from the group consisting of hydrogen, methyl, ethyl, —CH₂—OH, —(CH₂)ₙ—N[(C₁-C₂)alkyl]₂ with n being 2 or 3, —(CH₂)—OPO—[O(C₁-C₄)alkyl]_{z} and —(CH₂)—OC(═O)—(C₁-C₄)alkyl.

More preferably, the compound of formula (I) or (II), still more preferably of formula (II), has one or several of the following features:
R1 is a radical —O—(CH₂)ₙ—NRₐR_{b} with n being 2 or 3 or a radical —O—CH₂—(CHCH₃)—CH₂—NRₐR_{b}, wherein Rₐ and R_{b}, each independently, are selected from the group consisting of hydrogen and a (C₁-C₂)alkyl; or NRₐR_{b} may be taken together to form a heterocycle selected from the group consisting of piperidine and morpholine; and/or
R2 is hydrogen; and/or
R3 is selected from the group consisting of hydrogen, methyl and ethyl, and R4 is hydrogen; and/or
in formula (I), R5 is methoxy; and/or
in formula (II), X is an oxygen; and/or
in formula (II), R6 is hydrogen.

Still more preferably, the compound of formula (I) or (II), even more preferably of formula (II), has the following features:
R1 is a radical —O—(CH₂)ₙ—NRₐR_{b} with n being 2 or 3 or a radical —O—CH₂—(CHCH₃)—CH₂—NRₐR_{b}, wherein Rₐ and R_{b}, each independently, are selected from the group consisting of hydrogen and a (C₁-C₂)alkyl; or NRₐR_{b} may be taken together to form a heterocycle selected from the group consisting of piperidine and morpholine;
R2 is hydrogen;
R3 is selected from the group consisting of methyl and ethyl, and R4 is hydrogen; and

either
in formula (I), R5 is methoxy; or
in formula (II), X is an oxygen and R6 is hydrogen.

In a particularly preferred embodiment, the compound of formula (II) has the following features:
R1 is a radical —O—(CH₂)ₙ—NRₐR_{b} with n being 2 or 3 or a radical —O—CH₂—(CHCH₃)—CH₂—NRₐR_{b}, wherein Rₐ and R_{b}, each independently, are selected from the group consisting of hydrogen and a (C₁-C₂)alkyl; or NRₐR_{b} may be taken together to form a heterocycle selected from the group consisting of piperidine and morpholino;
R2 is hydrogen;
R3 is selected from the group consisting of methyl and ethyl, and R4 is hydrogen; and
X is an oxygen and R6 is hydrogen.

In a particular embodiment of the compound of formula (I) or (II) as defined above, R1 is —O—(CH₂)ₙ—N(CH₃)₂ or —O—(CH₂)ₙ—N(CH₂CH₃)₂ with n being 2 or 3. Preferably, R1 is —O—(CH₂)₂—N(CH₃)₂. More preferably, the compound is of formula (II) with X being an oxygen and R6 being hydrogen.

In a first very particular embodiment, the compound has the formula (II) with R1 being —O—(CH₂)₂—N(CH₃)₂ (preferably at position 3 of the benzo cycle A), R2, R4 and R6 being hydrogen, X being oxygen and R3 being methyl. In a second very particular embodiment, the compound has the formula (II) with R1 being —O—(CH₂)₂—N(CH₃)₂ (preferably at position 3 of the benzo cycle A), R2, R4 and R6 being hydrogen, X being oxygen and R3 being ethyl. In a third very particular embodiment, the compound has the formula (II) with R1 being 2-(morpholin-4-yl)ethoxy (preferably at position 3 of the benzo cycle A), R2, R4 and R6 being hydrogen, X being oxygen and R3 being methyl. In a fourth very particular embodiment, the compound has the formula (II) with R1 being 2-(morpholin-4-yl)ethoxy (preferably at position 3 of the benzo cycle A), R2, R4 and R6 being hydrogen, X being oxygen and R3 being ethyl. In a fifth very particular embodiment, the compound has the formula (II) with R1 being 2-(piperidin-1-yl)ethoxy (preferably at position 3 of the benzo cycle A), R2, R4 and R6 being hydrogen, X being oxygen and R3 being methyl. In a sixth very particular embodiment, the compound has the formula (II) with R1 being 2-(piperidin-1-yl)ethoxy (preferably at position 3 of the benzo cycle A), R2, R4 and R6 being hydrogen, X being oxygen and R3 being ethyl.

In a very particular aspect, the compound is selected from the group consisting of
11-M ethoxy-3-(2-N,N-dimethylamino ethoxy)-8-methyl-7H-benzo(e)pyrido(4,3-b)indole;
3-(2-N,N-Dimethylaminoethoxy)-8-methyl-7H,10H-benzo(e)pyrido(4,3-b)indol-11-one (C14);
3-(3-N,N-Dimethylaminopropoxy)-8-methyl-7H,10H-benzo(e)pyrido(4,3-b)indol-11-one;
3-(2-N,N-Diethylamino ethoxy)-8-methyl-7H,10H-benzo(e)pyrido(4,3-b)indol-11-one;
3-(2-(Morpholin-4-yl)ethoxy)-8-methyl-7H,10H-benzo(e)pyrido(4,3-b)indol-11-one (C07);
3-(2-(Morpholin-4-yl)ethoxy)-8-ethyl-7H,10H-benzo(e)pyrido(4,3-b)indol-11-one;
DL 3-(3-N,N-dimethylamino-2-methylpropoxy)-8-methyl-7H-10H-benzo(e)pyrido(4,3-b)indol-11-one;
3-(3-(Piperidin-1-yl)propoxy)-8-methyl-7H,10H-benzo(e)pyrido(4,3-b)indol-11-one;
3-(2-(Piperidin-1-yl)ethoxy)-8-methyl-7H,10H-benzo(e)pyrido(4,3-b)indol-11-one (C710);
3-(2-(Piperidin-1-yl)ethoxy)-8-ethyl-7H,10H-benzo(e)pyrido(4,3-b)indol-11-one;
3-(2-N,N-dimethylamino ethoxy)-8-ethyl-7H,11H-benzo(e)pyrido(4,3-b)indol-11-one (C48);
   and a pharmaceutically acceptable salt thereof, preferably a maleate salt thereof.

More preferably, the compound is selected from the group consisting of
3-(2-N,N-Dimethylaminoethoxy)-8-methyl-7H,10H-benzo(e)pyrido(4,3-b)indol-11-one (C 14);
3-(2-(Morpholin-4-yl)ethoxy)-8-methyl-7H,10H-benzo(e)pyrido(4,3-b)indol-11-one (C07);
3-(2-(Morpholin-4-yl)ethoxy)-8-ethyl-7H,10H-benzo(e)pyrido(4,3-b)indol-11-one;
3-(2-N,N-dimethylamino ethoxy)-8-ethyl-7H,11H-benzo(e)pyrido(4,3-b)indol-11-one (C48);
3-(2-(Piperidin-1-yl)ethoxy)-8-methyl-7H,10H-benzo(e)pyrido(4,3-b)indol-11-one (C710);
3-(2-(Piperidin-1-yl)ethoxy)-8-ethyl-7H,10H-benzo(e)pyrido(4,3-b)indol-11-one;
   and a pharmaceutically acceptable salt thereof, preferably a maleate salt thereof.

The present invention also relates to a pharmaceutical composition comprising a compound of the present invention and a pharmaceutically acceptable carrier. Optionally, the pharmaceutical composition may further comprise an additional antitumoral drug. Preferably, the additional antitumoral drug is a DNA-damaging anti-tumoral agent, preferably selected from the group consisting of an inhibitor of topoisomerase I or II, a DNA crosslinker, a DNA alkylating agent, and an anti-metabolic agent, preferably from the group consisting of an inhibitor of topoisomerases I and/or II and a DNA crosslinker.

The present invention further relates to a compound of the present invention as a drug. In particular, it relates to a compound of the present invention for use for treating cancer, inflammation, pain or parasitic infection, preferably for use for treating cancer. Optionally, the compound is for use for treating cancer in combination with radiotherapy, hyperthermia and/or an antitumoral chemotherapy, preferably a chemotherapy with a DNA-damaging anti-tumoral agent, more preferably a DNA-damaging anti-tumoral agent selected from the group consisting of an inhibitor of topoisomerase I or II, a DNA crosslinker, a DNA alkylating agent, and an anti-metabolic agent, preferably from the group consisting of an inhibitor of topoisomerases I and/or II and a DNA crosslinker. Alternatively, the present invention relates to a compound of the present invention as an antiparasitic agent. In addition, the present invention relates to a compound of the present invention for use for treating or preventing inflammation or pain.

In addition, the present invention relates to a kit or product comprising (a) a compound of the present invention; and (b) an additional antitumoral drug, preferably a DNA-damaging anti-tumoral agent, more preferably an anti-tumoral agent selected from the group consisting of an inhibitor of topoisomerase I or II, a DNA crosslinker, a DNA alkylating agent, and an anti-metabolic agent, preferably from the group consisting of an inhibitor of topoisomerases I and/or II and a DNA crosslinker, as a combined preparation for simultaneous, separate or sequential use, in particular in the treatment of cancer.

The present invention relates to a method for preparing a compound of formula (I)

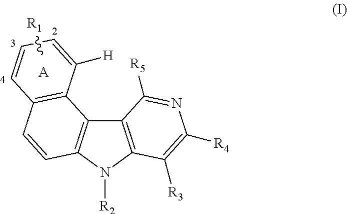

wherein R1, R2, R3, R4 and R5 are as defined in the present disclosure;

comprising

a) reacting the reagent Y—(C₂-C₅)alkyl-NRₐR_{b} with the compound 1

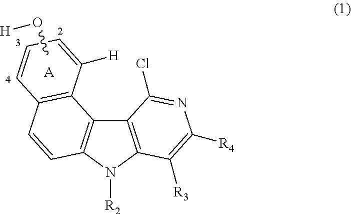

wherein Y is halo, or hydroxy, Rₐ, R_{b}, R2, R3 and R4 are as defined in the present disclosure;

and

b) reacting the compound obtained at step a) with alkali (C1-C4)alkoxide, thereby obtaining the compound of formula (I).

The present invention also relates to a method for preparing a compound of formula (II)

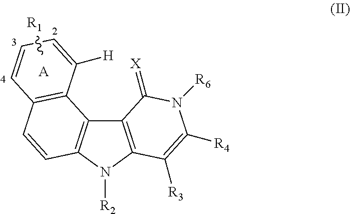

wherein R1, R2, R3, R4, R6 and X are as defined in the present disclosure;

comprising

a) reacting the reagent Y—(C₂-C₅)alkyl-NRₐR_{b} with the compound 1,

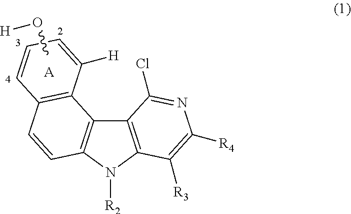

wherein Y is halo or hydroxy, Rₐ, R_{b}, R2, R3, R4 and R6 are as defined in the present disclosure;

and

b) reacting the compound obtained at step a) either with carboxylic acid anhydride, or with alkali carboxylate in carboxylic acid, thereby obtaining the compound of formula (II).

### BRIEF DESCRIPTION OF THE DRAWINGS

FIG. 1: Kinase profiling of compound C14

FIG. 1A: The potential inhibition of C14 was tested against 121 kinases, in vitro, at the concentration of 1 μM. The kinase activities, in the presence of C14, are represented, each kinase being represented by a vertical bar. Only 18 kinases have less than 30% activity in the presence of C14.

FIG. 1B: comparison of the main C14 targets (kinases inhibited by more than 70%) with CH21 targets. Main CH21 targets (kinases inhibited by more than 50%) are indicated by a star. The percent of activity of each kinase is represented by an histogram (grey for CH21 and white for C 14).

FIG. 2: Comparison of the efficiency of C14, C1 and CH21 towards few targets.

Assays were run in vitro against recombinant kinases. The activity is reported for each kinase, grey histograms for CH21, white for C14 and gradation grey for C1. None of these kinases were significantly affected by CH21. Both RSK1, 2, Src and Aurora A were not affected by C14, although they are highly inhibited by C1. Therefore, C1 and C14 affect differently the Aurora kinase family.

FIG. 3. Inhibition of Aurora B, in U2OS cells

The intensity of Histone H3 phosphorylation (Histone H3-P) reveals Aurora B activity. Actin is used as control of quantity. Cells were incubated overnight in the presence of either C1, or C2 or CH21 or C14 or DMSO(C: control). All compounds were tested at 1 μM. Proteins were detected by western blotting.

FIG. 4: Inhibition of Aurora B by benzo[e]pyridoindoles

Cells were incubated with the different molecules and Aurora B inhibition is estimated by western blotting (WB) and immuno fluorescent experiments. Histone H3 phosphorylated on Ser10 is used as a marker of Aurora B kinase activity and actin as an internal control. Each WB signal is compared to the control in the absence of compound. The efficiency estimated by WB is indicated by +++: very efficient inhibition to −: no inhibition. Immunofluorescent labelling of a mitotic cell by Histone H3 illustrates Aurora B kinase activity. However immunofluorescence is less sensitive than WB. Active molecules are underlined by a square and the intensity of the line indicates the efficiency of inhibition.

FIG. 5: Quiescent H358 cells were incubated with the compound, for 96 h, in the absence of serum. Three different concentrations of C14 were used and live cells were estimated with MTT (Promega). In the control only DMSO was added.

FIG. 6: Analysis of cell cycle by FACS

The influence of the C14 treatment versus C1 and control (Te) were analysed by FACS following propidium iodine labelling. C1 and C14 were added at the concentration of 0.5 μM and the treatment lasted either 30 h or 48 h. On the top histogram, arrows allowed the visualization of the different phases of the cell cycle and the corresponding data are reported in Table 4.

FIG. 7: Time-lapse of HeLa cells stably expressing aurora B-GFP

FIG. 7A: Cells treated by either DMSO (CO: control) or C1 (0.5 μM) or C14 (0.5 μM) were continuously imaged with a ZEISS confocal microscope. GFP revealed the presence and the localisation of aurora B kinase. Mitotic cells were found fluorescent and GFP was concentrated on the centromeres. In control conditions, centromere aligned at metaphase and upon anaphase ongoing fluorescence concentrates on the spindle and finally in the midbody (A, CO). The shape of the cells is visualized by white dotted lines.

In the presence of C 1, centromeres were never found aligned (FIG. 7A, C1). In the presence of C14, centromeres aligned normally and fluorescence concentrated on the mid-zone but cells failed to separate (see the shape of the cells indicated by white dotted lines and images under each fluorescent photo).

FIG. 7B: Nuclei of cells treated as above (FIG. 7A) were labelled with Hoechst. Note the presence of connecting chromatin in already decondensed nuclei (C14a) that give rise to two contacting nuclei. Treatments are indicated on each image and C0: control.

FIG. 7C: Visualisation of C1 and C14 actions in mitosis.

Both C 1 and C14 prevented mitosis ongoing but C1 altered spindle checkpoint whereas C14 impairs cell segregation.

FIG. 8 Time-lapses of Hek cells expressing tubulin-GFP

Mitotic Hek cells stably expressing tubulin-GFP were continuously imaged and representative of either control or treated cells (C14 and C1, 0.5 μM) are shown. Elapse times are indicated on each photo. C14 prevented mitotic spindle organization whereas C1 induced its duplication.

FIG. 9: Comparison of compound efficiencies on spheroid growth.

H358 cells were cultured in 3-Dimensions. At day 1, the compound was added at the concentration of 0.6 μM. The spheroid growth was measured each day and expressed as a growth ratio (Vd−Vo/Vo); d for day and o for the starting day. The evolution of the volume of the spheroids is expressed in function of the time (in days). Spheroids grown in the presence of C1 are indicated by triangles whereas rectangles indicate spheroids treated by C14; control spheroids are visualized by diamonds.

FIG. 10: Efficiency of different molecules on Histone H3 phosphorylation on Ser 10 immunofluorescent assay.

FIG. 10A: Immunofluorescence with phospho-Ser10-Histone H3 antibodies. Either the percentage of positive phospho-Ser10-Histone H3 is indicated or a representative mitosis shown. (+++) indicated a strong inhibition and no positive mitosis detected whereas (−) revealed no difference with the control. Immunofluorescent assays were realized with a specific phospho-antibody and mitotic U2OS cells are recognized by their DNA content stained with Hoechst. On the right, a representative cell or the percentage of H3-S10-P labelled mitosis.

FIG. 10B: Immunoblotting analysis of histone H3 phosphorylation. U2OS cells were incubated overnight with Nocodazole (50 nM) in the presence of either DMSO (Co) or Compounds (1 μM). Cells were then collected and lyzed. Identical amounts of the lyzed cell samples were run on 18% acrylamide gel containing SDS. After transfer, the blot was revealed using an antibody against phosphorylated histone H3 (H3-S10-P). The same membrane was also revealed using antibodies against α-tubulin and Aurora B for estimating both the amount of loaded proteins and of mitotic cells, respectively.

FIG. 10C: Immunoblotting analysis of histone H3 phosphorylation. U2OS cells were incubated overnight with Nocodazole (50 nM) in the presence of either DMSO (Co) or 3-(2-(Morpholin-4-yl)ethoxy) Compounds (C07 and C05, 1 μM) or 3-(2-(pyperidin-1-yl)ethoxy) Compounds (C710 and C709, 1 μM). Cells were then collected and lyzed. Identical amounts of the lyzed cell samples were run on 18% acrylamide gel containing SDS. After transfer, the blot was revealed using an antibody against phosphorylated histone H3 (H3-S10-P). The same membrane was also revealed using antibodies against Aurora B for estimating the amount of mitotic cells.

FIG. 10D: Immunoblotting analysis of histone H3 phosphorylation. U2OS cells were incubated overnight with Nocodazole (50 nM) in the presence of either DMSO (Co) or 3-(2-methoxyethoxy) Compounds (C708 et C711, 1 μM). Cells were then collected and lyzed. Identical amounts of the lyzed cell samples were run on 18% acrylamide gel containing SDS. After transfer, the blot was revealed using an antibody against phosphorylated histone H3 (H3-S10-P). The same membrane was also revealed using antibodies against Aurora B for estimating the amount of mitotic cells.

FIG. 11: Validation of the corresponding maleate salts.

FIG. 11A: The maleate salts corresponding to aurora kinase B inhibitors were synthesized. The molecules noted C14M, C48M and C07M are represented.

FIGS. 11B and 11C: Immunoblotting analysis of histone H3 phosphorylation. U2OS and HeLa cells were incubated overnight with Nocodazole (50 nM) in the presence of either DMSO (Co) or Compounds (1 μM). Cells were then collected and lyzed. Identical amounts of the lyzed cell samples were run on 18% acrylamide gel containing SDS. After transfer, the blot was revealed using an antibody against phosphorylated histone H3 (H3-S10-P). The same membrane was also revealed using antibodies against Aurora B for estimating the amount of mitotic cells.

FIG. 12: Quantification of LATS1 upon C14M treatment

HeLa cells were treated ON by C21 (also named CH21) and C14M and compared to the control (C0). Whole cell extracts were analyzed by WB with anti LATS1 monoclonal antibody and the loading was quantified through the tubulin signal. Note the large increase of LATS1 upon treatment.

### DETAILED DESCRIPTION OF THE INVENTION

The inventors identified a new class of benzo[e]pyrido-indoles, the amino-substituted-alkyloxy-benzo[e]pyrido[4,3-b]indole derivatives. This new class of compounds presents a therapeutic interest, in particular as an antiproliferative drug with their broad anti-proliferating activities. They are structurally characterized by the presence on the benzo cycle A of a group aminoalkoxy and at position 11 of a group oxo or alkoxy. Indeed, those features provide to the compounds an Aurora B inhibitory specificity in comparison to Aurora A. Due to the targeting of MELK, they may also be proposed for cancer stem cells targeting (e.g., neuroblastoma, colorectal cancers, lung and breast cancer). In addition, by their capacity to inhibit checkpoint kinases Chk-1 and Chk-2, it supports the benefit of their combined use with DNA damaging agents. Indeed, the double targeting of aurora B and chk2 kinases by these compounds acts cooperatively to kill cancer cells since cells entering in mitosis with damaged DNA are highly susceptible to cell death. In addition, the compounds of the present invention inhibit Gck, allowing anti-tumoral and anti-inflammatory effects; and they inhibit TrkA, being an oncogene and playing a role in chronic inflammatory pain. The compounds of the present invention target ARK5/Nuak1, a key signaling kinase in tumour cells that express deregulated Myc (Liu et al, 2012, Nature 483, 608-612). The compounds of the invention present the additional advantage to be soluble in water.

In particular, the compounds of the present invention have a kinase inhibition profile distinct from compounds C1 and C2 (Hoang et al, 2009), leading to biological activity distinct from them. Indeed, the new class of the present invention surprisingly presents an inhibition profile which is more specific of Aurora B in comparison to Aurora A, whereas C1 and C2 compounds inhibit both Aurora A and B. This specificity leads to an inhibition of the cell cycle at later stages than C1. In particular, the Aurora B inhibitory activity of C14 is higher than the compound C1. Then, the inventors surprisingly identified the functional impact of the structural modification on the benzo cycle A with an amino-alkyl. In addition, in comparison with compounds C1 and C2 (Hoang et al, 2009), the compounds of the present invention have a higher antiproliferative efficiency.

Therefore, the present invention relates to a compound having the formula (I) or (II)

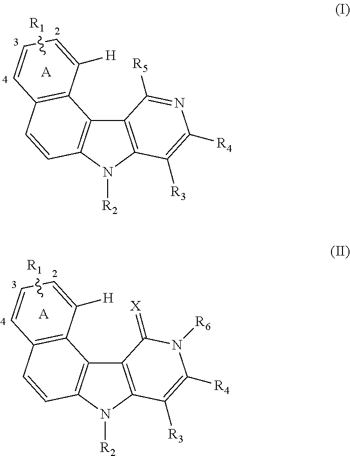

wherein

the benzo cycle A is mono-substituted by R1 in position 2, 3 or 4;

R1 is a radical —O—(C₂-C₅)alkyl-NRₐR_{b}, wherein (C₂-C₅)alkyl is a linear or branched alkyl, Rₐ and R_{b}, each independently, are selected from the group consisting of hydrogen, a (C₁-C₄)alkyl optionally substituted by a radical selected from the group consisting of hydroxyl, —NRR′, OPO(OR)(OR′), and —OC(═O)R, and a (C₃-C₆)cycloalkyl; or NRₐR_{b} may be taken together to form a heterocycle selected from aziridine, azetidine, pyrrolidine, pyrrole, piperidine, piperazine, morpholine, thiomorpholine, the said heterocycle is optionally substituted by a (C₁-C₄)alkyl or hydroxyl radical;

R2 is selected from the group consisting of hydrogen and a (C₁-C₃)alkyl optionally substituted by a radical OH, (C₁-C₃)alkyloxy or —NRR′,

R3 and R4, each independently, are selected from the group consisting of hydrogen, a (C1-C3)alkyl and an aryl; or R3 and R4 may be taken together to form a bivalent radical of formula
—(CH₂)ₙ— wherein n is 3, 4 or 5; or
—CH═CH—CH═CH—, optionally substituted by a (C₁-C₃)alkyloxy;

R5, only present in formula (I), is a (C₁-C₄)alkoxy;

R6, only present in formula (II), is selected from the group consisting of hydrogen and a (C₁-C₃)alkyl, optionally substituted by a radical selected from the group consisting of hydroxyl, —NRR′, —OPO(OR)(OR′), and —OC(═O)R;

X, only present in formula (II), is O or S;

wherein R and R′, identical or different, are selected from the group consisting of hydrogen and (C₁-C₄)alkyl, preferably hydrogen and (C₁-C₂)alkyl;

or an isomeric form thereof or a pharmaceutically acceptable salt thereof or a derivative thereof.

In the context of the present invention, the term “(C₁-C₂)alkyl” more specifically means methyl or ethyl, the term “(C₁-C₃)alkyl” more specifically means methyl, ethyl, propyl, or isopropyl and “(C₁-C₄)alkyl” more specifically means methyl, ethyl, propyl, isopropyl, butyl, isobutyl, or tert-butyl, the term “(C₂-C₅)alkyl” more specifically means methyl, ethyl, propyl, isopropyl, butyl, isobutyl, tert-butyl or propyl.

“Alkoxy” groups correspond to the alkyl groups defined hereinabove bonded to the molecule by an —O— (ether) bond. (C₁-C₃)alkoxy includes methoxy, ethoxy, propyloxy, and isopropyloxy. (C₁-C₄)alkoxy includes methoxy, ethoxy, propyloxy, isopropyloxy, butyloxy, isobutyloxy, and tert-butyloxy.

The “aryl” or “Ar” group is mono- or bi-cyclic aromatic hydrocarbons having from 6 to 12 carbon atoms, optionally substituted. Aryl may be a phenyl, biphenyl or naphthyl. In a preferred embodiment, the aryl is a phenyl.

The term “derivative” is meant to encompass hydrate, ester, ether, conjugates, or pro-drugs thereof. For instance, the compounds with a radical —OPO(OR)(OR′) as defined above is a prodrug and has an increased solubility.

(C₃-C₆)cycloalkyl includes cyclopropyl, cyclobutyl, cyclopentyl and cyclohexyl.

“Halogeno” or “halo” groups are preferably selected from the group consisting of Cl (chloride), Br (bromide), I (iodide) and F (fluoride).

The pharmaceutically acceptable salts include salts of inorganic acids as well as organic acids. Representative examples of suitable inorganic acids include hydrochloric, hydrobromic, hydroiodic, phosphoric, and the like. Representative examples of suitable organic acids include formic, acetic, trichloroacetic, trifluoroacetic, propionic, benzoic, cinnamic, citric, fumaric, maleic, methanesulfonic and the like. Further examples of pharmaceutically acceptable inorganic or organic acid addition salts include the pharmaceutically acceptable salts listed in J. Pharm. Sci. 1977, 66, 2, and in Handbook of Pharmaceutical Salts: Properties, Selection, and Use edited by P. Heinrich Stahl and Camille G. Wermuth 2002. In a preferred embodiment, the salt is selected from the group consisting of maleate, chlorhydrate, bromhydrate, and methanesulfonate. In a specific embodiment, the salt is maleate.

In a preferred embodiment, R1 is a radical —O—(CH₂)ₙ—NRₐR_{b} with n being 2 or 3 or a radical —O—CH₂—(CHCH₃)—CH₂—NRₐR_{b}, wherein Rₐ and R_{b}, each independently, are selected from the group consisting of hydrogen and a (C₁-C₂)alkyl; or NRₐR_{b} may be taken together to form a heterocycle selected from the group consisting of piperidine and morpholino. More preferably, R1 is a radical —O—(CH₂)ₙ—NRₐR_{b} with n being 2 or 3 or a radical —O—CH₂—(CHCH₃)—CH₂—NRₐR_{b}, wherein Rₐ and R_{b}, each independently, are selected from the group consisting of hydrogen and (C₁-C₂)alkyl; or NRₐR_{b} may be taken together to form a heterocycle selected from the group consisting of piperidine and morpholino. Preferably, Rₐ and R_{b}, each independently, are selected from the group consisting of methyl and ethyl. In a particular embodiment of the compound of formula (I) or (II) as defined above, R₁ is —O—(CH₂)ₙ—N(CH₃)₂ or —O—(CH₂)ₙ—N(CH₂CH₃)₂ with n being 2 or 3. Preferably, R₁ is —O—(CH₂)₂—N(CH₃)₂. In the most preferred embodiment, R1 is —O—(CH₂)₂—N(CH₃)₂. In a second most preferred embodiment, R1 is 2-(morpholin-4-yl)ethoxy. In a third most preferred embodiment, R1 is 2-(piperidin-1-yl)ethoxy. More preferably, R1 is at position 3 of the benzo cycle A.

In a preferred embodiment, R2 is selected from the group consisting of hydrogen and a (C₁-C₃)alkyl, more preferably is hydrogen.

In a preferred embodiment, R3 and R4, each independently, are selected from the group consisting of hydrogen, a (C₁-C₃)alkyl and an aryl. More preferably, R3 is selected from the group consisting of a (C₁-C₃)alkyl and an aryl, and R4 is selected from the group consisting of hydrogen and a (C₁-C₃)alkyl. Still more preferably, R3 is a (C₁-C₃)alkyl, and R4 is hydrogen.

In a preferred embodiment, in formula (I), R5 is a (C₁-C₃)alkoxy, more preferably is methoxy or ethoxy, still more preferably is methoxy.

In a preferred embodiment, in formula (II), R6 is selected from the group consisting of hydrogen and a (C₁-C₃)alkyl, more preferably is hydrogen. In addition, X is preferably oxygen.

Preferably, the compound of formula (I) or (II), more preferably of formula (II), may have one or several of the following features:
R2 is hydrogen; and/or
R3 is selected from the group consisting of hydrogen, methyl and ethyl, and R4 is hydrogen; and/or
in formula (I), R5 is methoxy; and/or
in formula (II), X is an oxygen; and/or, R6 is hydrogen.

Still more preferably, the compound of formula (I) or (II), even more preferably of formula (II), may have one or several of the following features:
R2 is hydrogen; and/or
R3 is selected from the group consisting of methyl and ethyl; and/or
R4 is hydrogen; and/or

either
in formula (I), R5 is methoxy; or
in formula (II), X is an oxygen and R6 is hydrogen.

Even more preferably, the compound of formula (I) or (II), still even more preferably of formula (II), may have the following features:
R2 is hydrogen;
R3 is selected from the group consisting of methyl and ethyl, and R4 is hydrogen; and

either
in formula (I), R5 is methoxy; or
in formula (II), X is an oxygen and R6 is hydrogen.

In a particularly preferred embodiment, the compound of formula (II) has the following features:
R1 is a radical —O—(CH₂)ₙ—NRₐR_{b} with n being 2 or 3 or a radical —O—CH₂—(CHCH₃)—CH₂—NRₐR_{b}, wherein Rₐ and R_{b}, each independently, are selected from the group consisting of hydrogen and a (C₁-C₂)alkyl; or NRₐR_{b} may be taken together to form a heterocycle selected from the group consisting of piperidine and morpholine;
R2 is hydrogen;
R3 is selected from the group consisting of methyl and ethyl, and R4 is hydrogen; and
X is an oxygen and R6 is hydrogen.

In a first particular embodiment, the compound has the formula (II) with R1 being —O—(CH₂)ₙ—N(CH₃)₂ (preferably at position 3 of the benzo cycle A), n being 2 or 3, R2, R4 and R6 being hydrogen, X being oxygen and R3 being methyl or ethyl. More specifically, n is 2. In a first embodiment, R3 is methyl. In a second embodiment, R3 is ethyl.

In a second very particular embodiment, the compound has the formula (I) with R1 being —O—(CH₂)ₙ—N(CH₃)₂ (preferably at position 3 of the benzo cycle A), n being 2 or 3, R2 being hydrogen, R5 being methoxy and R3 being methyl or ethyl. More specifically, n is 2. In a first embodiment, R3 is methyl. In a second embodiment, R3 is ethyl.

In a third very particular embodiment, the compound has the formula (II) with R1 being —O—(CH₂)ₙ—N(CH₂CH₃)₂ (preferably at position 3 of the benzo cycle A), n being 2 or 3, R2, R4 and R6 being hydrogen, X being oxygen and R3 being methyl or ethyl. More specifically, n is 2. In a first embodiment, R3 is methyl. In a second embodiment, R3 is ethyl.

In a fourth very particular embodiment, the compound has the formula (I) with R1 being —O—(CH₂)ₙ—N(CH₂CH₃)₂ (preferably at position 3 of the benzo cycle A), n being 2 or 3, R2 being hydrogen, R5 being methoxy and R3 being methyl or ethyl. More specifically, n is 2. In a first embodiment, R3 is methyl. In a second embodiment, R3 is ethyl.

In a fifth very particular embodiment, the compound has the formula (II) with R1 being —O—(CH₂)ₙ-morpholino (preferably at position 3 of the benzo cycle A), n being 2 or 3, R2, R4 and R6 being hydrogen, X being oxygen and R3 being methyl or ethyl. More specifically, n is 2. In a first embodiment, R3 is methyl. In a second embodiment, R3 is ethyl.

In a sixth very particular embodiment, the compound has the formula (I) with R1 being —O—(CH₂)₂-morpholino (preferably at position 3 of the benzo cycle A), n being 2 or 3, R2 being hydrogen, R5 being methoxy and R3 being methyl or ethyl. More specifically, n is 2. In a first embodiment, R3 is methyl. In a second embodiment, R3 is ethyl.

In a seventh very particular embodiment, the compound has the formula (II) with R1 being O—CH₂—(CHCH₃)—CH₂—N(CH₃)₂ (preferably at position 3 of the benzo cycle A), R2, R4 and R6 being hydrogen, X being oxygen and R3 being methyl or ethyl. In a first embodiment, R3 is methyl. In a second embodiment, R3 is ethyl.

In a eighth particular embodiment, the compound has the formula (I) with R1 being —O—CH₂—(CHCH₃)—CH₂—N(CH₃)₂ (preferably at position 3 of the benzo cycle A), R2 being hydrogen, R5 being methoxy and R3 being methyl or ethyl. In a first embodiment, R3 is methyl. In a second embodiment, R3 is ethyl.

In a ninth very particular embodiment, the compound has the formula (II) with R1 being —O—(CH₂)ₙ-piperidin-1-yl (preferably at position 3 of the benzo cycle A), n being 2 or 3, R2, R4 and R6 being hydrogen, X being oxygen and R3 being methyl or ethyl. More specifically, n is 2. In a first embodiment, R3 is methyl. In a second embodiment, R3 is ethyl.

In an additional particular embodiment, the compound has the formula (I) with R1 being —O—(CH₂)ₙ-piperidin-1-yl (preferably at position 3 of the benzo cycle A), n being 2 or 3, R2 being hydrogen, R5 being methoxy and R3 being methyl or ethyl. More specifically, n is 2. In a first embodiment, R3 is methyl. In a second embodiment, R3 is ethyl.

In a preferred embodiment of the invention, the compounds are of formula (II).

Optionally, the compound of the invention may be selected from the group consisting of
11-Methoxy-3-(2-N,N-dimethylaminoethoxy)-8-methyl-7H-benzo(e)pyrido(4,3-b)indole;
3-(2-N,N-Dimethylaminoethoxy)-8-methyl-7H,10H-benzo(e)pyrido(4,3-b)indol-11-one;
3-(3-N,N-Dimethylaminopropoxy)-8-methyl-7H,10H-benzo(e)pyrido(4,3-b)indol-11-one;
3-(2-N,N-Diethylamino ethoxy)-8-methyl-7H,10H-benzo (e) pyrido (4,3-b)indol-11-one;
3-(2-(Morpholin-4-yl)ethoxy)-8-methyl-7H,10H-benzo(e)pyrido (4,3-b)indol-11-one;
3-(2-(Morpholin-4-yl)ethoxy)-8-ethyl-7H,10H-benzo(e)pyrido (4,3-b)indol-11-one;
DL 3-(3-N,N-dimethylamino-2-methylpropoxy)-8-methyl-7H-10H-benzo[e]pyrido(4,3-b)indol-11-one;
3-(3-(Piperidin-1-yl)propoxy)-8-methyl-7H,10H-benzo(e)pyrido (4,3-b)indol-11-one;
3-(2-(Piperidin-1-yl)ethoxy)-8-methyl-7H,10H-benzo(e)pyrido (4,3-b)indol-11-one;
3-(2-(Piperidin-1-yl)ethoxy)-8-ethyl-7H,10H-benzo(e)pyrido (4,3-b)indol-11-one;
3-(2-N,N-dimethylamino ethoxy)-8-ethyl-7H,11H-benzo(e)pyrido(4,3-b)indol-11-one;
   and a pharmaceutically acceptable salt thereof.

More preferably, it selected from the group consisting of
11-M ethoxy-3-(2-N,N-dimethylamino ethoxy)-8-methyl-7H-benzo(e)pyrido(4,3-b)indole;
3-(2-N,N-Dimethylaminoethoxy)-8-methyl-7H,10H-benzo(e)pyrido(4,3-b)indol-11-one;
3-(3-N,N-Dimethylaminopropoxy)-8-methyl-7H,10H-benzo(e)pyrido(4,3-b)indol-11-one;
3-(2-N,N-dimethylamino ethoxy)-8-ethyl-7H,11H-benzo(e)pyrido(4,3-b)indol-11-one;
   and a pharmaceutically acceptable salt thereof.

In another preferred embodiment, it selected from the group consisting of
3-(2-N,N-Dimethylaminoethoxy)-8-methyl-7H,10H-benzo(e)pyrido(4,3-b)indol-11-one;
3-(3-N,N-Dimethylaminopropoxy)-8-methyl-7H,10H-benzo(e)pyrido(4,3-b)indol-11-one;
3-(2-N,N-dimethylamino ethoxy)-8-ethyl-7H,11H-benzo(e)pyrido(4,3-b)indol-11-one;
3-(3-N,N-Dimethylaminopropoxy)-8-ethyl-7H,10H-benzo(e)pyrido(4,3-b)indol-11-one;
3-(2-(Morpholin-4-yl)ethoxy)-8-methyl-7H,10H-benzo[e]pyrido(4,3-b)indol-11-one;
3-(2-(Morpholin-4-yl)ethoxy)-8-ethyl-7H,10H-benzo[e]pyrido(4,3-b)indol-11-one;
3-(2-(Piperidin-1-yl)ethoxy)-8-methyl-7H,10H-benzo[e]pyrido(4,3-b)indol-11-one;
3-(2-(Piperidin-1-yl)ethoxy)-8-ethyl-7H,10H-benzo[e]pyrido(4,3-b)indol-11-one;
   and a pharmaceutically acceptable salt thereof.

In a more preferred embodiment, it selected from the group consisting of
3-(3-N,N-Dimethylaminopropoxy)-8-methyl-7H,10H-benzo(e)pyrido(4,3-b)indol-11-one;
3-(3-N,N-Dimethylaminopropoxy)-8-ethyl-7H,10H-benzo(e)pyrido(4,3-b)indol-11-one;
3-(2-(Morpholin-4-yl)ethoxy)-8-methyl-7H,10H-benzo[e]pyrido(4,3-b)indol-11-one;
3-(2-(Morpholin-4-yl)ethoxy)-8-ethyl-7H,10H-benzo[e]pyrido(4,3-b)indol-11-one;
3-(2-(Piperidin-1-yl)ethoxy)-8-methyl-7H,10H-benzo[e]pyrido(4,3-b)indol-11-one;
3-(2-(Piperidin-1-yl)ethoxy)-8-ethyl-7H,10H-benzo[e]pyrido(4,3-b)indol-11-one;
   and a pharmaceutically acceptable salt thereof.

In a more specific embodiment, it selected from the group consisting of
3-(3-N,N-Dimethylaminopropoxy)-8-methyl-7H,10H-benzo(e)pyrido(4,3-b)indol-11-one;
3-(3-N,N-Dimethylaminopropoxy)-8-ethyl-7H,10H-benzo(e)pyrido(4,3-b)indol-11-one;
3-(2-(Morpholin-4-yl)ethoxy)-8-methyl-7H,10H-benzo[e]pyrido(4,3-b)indol-11-one;
3-(2-(Piperidin-1-yl)ethoxy)-8-methyl-7H,10H-benzo[e]pyrido(4,3-b)indol-11-one;
   and a pharmaceutically acceptable salt thereof.

The present invention relates to
a pharmaceutical composition comprising a) any compound having the formula (I) or (II) as disclosed above including anyone of the disclosed embodiments, and a pharmaceutically acceptable carrier, in particular for use in the treatment of cancer, inflammation, pain or parasitic infection; and/or
any compound having the formula (I) or (II) as disclosed above including anyone of the disclosed embodiments as a drug, in particular an anti-tumoral drug, an anti-inflammatory drug, an anti-pain drug, or an anti-parasitic drug; and/or
a pharmaceutical composition comprising a) any compound having the formula (I) or (II) as disclosed above including anyone of the disclosed embodiments, b) an additional anti-tumoral agent, preferably a DNA-damaging anti-tumoral agent, and a pharmaceutically acceptable carrier, in particular for use in the treatment of cancer; and/or
a product or kit containing (a) any compound of formula (I) or (II) as disclosed above including anyone of the disclosed embodiments and (b) an additional anti-tumoral agent, preferably a DNA-damaging anti-tumoral agent, as a combined preparation for simultaneous, separate or sequential use, in particular in the treatment of cancer; and/or
a combined preparation which comprises (a) any compound of formula (I) or (II) as disclosed above including anyone of the disclosed embodiments and (b) an additional anti-tumoral agent, preferably a DNA-damaging anti-tumoral agent, for simultaneous, separate or sequential use, in particular in the treatment of cancer; and/or
a pharmaceutical composition comprising any compound having the formula (I) or (II) as disclosed above including anyone of the disclosed embodiments for the use in the treatment of cancer in combination with radiotherapy and/or hyperthermia; and/or
the use of a pharmaceutical composition comprising any compound having the formula (I) or (II) as disclosed above including anyone of the disclosed embodiments for the manufacture of a medicament for the treatment of cancer, inflammation, pain or parasitic infection, preferably cancer; and/or
the use of a pharmaceutical composition comprising any compound having the formula (I) or (II) as disclosed above including anyone of the disclosed embodiments for the manufacture of a medicament for the treatment of cancer in combination with radiotherapy, hyperthermia and/or an additional anti-tumoral agent, preferably a DNA-damaging anti-tumoral agent; and/or
the use of a pharmaceutical composition comprising a) any compound having the formula (I) or (II) as disclosed above including anyone of the disclosed embodiments and b) an additional anti-tumoral agent, preferably a DNA-damaging anti-tumoral agent, and a pharmaceutically acceptable carrier for the manufacture of a medicament for the treatment of cancer; and/or
a method for treating a cancer in a subject in need thereof, comprising administering an effective amount of a pharmaceutical composition comprising any compound having the formula (I) or (II) as disclosed above including anyone of the disclosed embodiments, and a pharmaceutically acceptable carrier; and/or
a method for treating a cancer in a subject in need thereof, comprising administering an effective amount of a pharmaceutical composition comprising a) any compound having the formula (I) or (II) as disclosed above including anyone of the disclosed embodiments, b) an additional anti-tumoral agent, preferably a DNA-damaging anti-tumoral agent, and a pharmaceutically acceptable carrier; and/or
a method for treating a cancer in a subject in need thereof, comprising administering an effective amount of a pharmaceutical composition comprising any compound having the formula (I) or (II) as disclosed above including anyone of the disclosed embodiments, and an effective amount of a pharmaceutical composition comprising an additional anti-tumoral agent, preferably a DNA-damaging anti-tumoral agent; and/or
a method for treating a cancer in a subject in need thereof, comprising administering an effective amount of a pharmaceutical composition comprising a) any compound having the formula (I) or (II) as disclosed above including anyone of the disclosed embodiments in combination with radiotherapy or hyperthermia; and/or
a method for treating an inflammation, pain or a parasitic infection in a subject in need thereof, comprising administering an effective amount of a pharmaceutical composition comprising any compound having the formula (I) or (II) as disclosed above including anyone of the disclosed embodiments, and a pharmaceutically acceptable carrier.

Whenever within this whole specification “treatment of a cancer” or the like is mentioned with reference to the pharmaceutical composition of the invention, there is meant: a) a method for treating a cancer, said method comprising administering a pharmaceutical composition of the invention to a subject in need of such treatment; b) the use of a pharmaceutical composition of the invention for the treatment of a cancer; c) the use of a pharmaceutical composition of the invention for the manufacture of a medicament for the treatment of a cancer; d) a pharmaceutical composition comprising a dose of any compound having the formula (I) or (II) as disclosed above including anyone of the disclosed embodiments and of an additional anti-tumoral agent, preferably a DNA-damaging anti-tumoral agent, that is appropriate for the treatment of a cancer; and/or e) a pharmaceutical composition of the invention for treating a cancer.

Pain includes acute pain, chronic pain, neuropathic pain, muscular pain, bone pain, postoperative pain, migraine, cancer-related pain, lumbalgia, arthrosic pain, diabetes-related pain or pain associated to AIDS. In a particularly preferred embodiment, pain is a cancer-related pain, especially a cancer with bone metastasis.

By “inflammation” or “inflammatory disorder or disease” refer to any disorder, condition, or disease characterized or caused by excessive or uncontrolled inflammation, or any aspect of inflammation such as redness, swelling, heat, pain, etc. In particular, it may refer to chronic or acute inflammation. Inflammatory diseases include, but are not limited to, irritable bowel disease, Crohn's disease, ulcerative colitis, allergies, including allergic rhinitis/sinusitis, skin allergies such as urticaria/hives, angioedema, atopic dermatitis, food allergies, drug allergies, insect allergies, and rare allergic disorders such as mastocytosisasthma, asthma, arthritis, including osteoarthritis, rheumatoid arthritis, and spondyloarthropathies, gastrointestinal inflammation, neuroinflammatory disorders, and autoimmune disorders.

By “parasitic infection” is intended to refer to a disease caused by protozoa (causing protozoan infection), helminths (helminthiasis), and ectoparasites.

The term “pharmaceutically acceptable carrier” is meant to encompass any carrier (e.g., support, substance, solvent, etc.) which does not interfere with effectiveness of the biological activity of the active ingredient(s) and that is not toxic to the host to which it is administered. For example, for parenteral administration, the active compounds(s) may be formulated in a unit dosage form for injection in vehicles such as saline, dextrose solution, serum albumin and Ringer's solution.

The pharmaceutical composition can be formulated as solutions in pharmaceutically compatible solvents or as emulsions, suspensions or dispersions in suitable pharmaceutical solvents or vehicule, or as pills, tablets or capsules that contain solid vehicules in a way known in the art. Formulations of the present invention suitable for oral administration may be in the form of discrete units as capsules, sachets, tablets or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion. Formulations for rectal administration may be in the form of a suppository incorporating the active ingredient and carrier such as cocoa butter, or in the form of an enema. Formulations suitable for parenteral administration conveniently comprise a sterile oily or aqueous preparation of the active ingredient which is preferably isotonic with the blood of the recipient. Every such formulation can also contain other pharmaceutically compatible and nontoxic auxiliary agents, such as, e.g. stabilizers, antioxidants, binders, dyes, emulsifiers or flavouring substances. The formulations of the present invention comprise an active ingredient in association with a pharmaceutically acceptable carrier therefore and optionally other therapeutic ingredients. The carrier must be “acceptable” in the sense of being compatible with the other ingredients of the formulations and not deleterious to the recipient thereof. The pharmaceutical compositions are advantageously applied by injection or intravenous infusion of suitable sterile solutions or as oral dosage by the digestive tract. Methods for the safe and effective administration of most of these chemotherapeutic agents are known to those skilled in the art. In addition, their administration is described in the standard literature.

Radiotherapy includes, but is not limited to, γ-rays, X-rays, and/or the directed delivery of radioisotopes to tumor cells. Other radiotherapies include microwaves and UV-irradiation. Other approaches to radiation therapy are also contemplated in the present invention. The radiotherapy may be applied before or simultaneously with the administration of the compound of the present invention. When the administration of the compound is after radiotherapy, the compound may be for instance administered 1, 2, 3, 4, 5, 6, 12, 18 or 24 h after the radiotherapy.

Hyperthermia is a medical treatment in which body tissue is exposed to high temperatures to damage and kill cancer cells or to make cancer cells more sensitive to the effects of radiation and certain anti-cancer drugs. There are many techniques, well-known by the on skilled in the art, by which heat may be delivered. Some of the most common involve the use of focused ultrasound (FUS or HIFU), infrared sauna, microwave heating, induction heating, magnetic hyperthermia, infusion of warmed liquids, or direct application of heat such as through sitting in a hot room or wrapping a patient in hot blankets.

The DNA-damaging anti-tumoral agent may be chosen from the group consisting of inhibitors of topoisomerases I and/or II, DNA crosslinkers, DNA alkylating agents, and anti-metabolic agents. In a preferred embodiment, the DNA-damaging anti-tumoral agent is chosen from the group consisting of inhibitors of topoisomerases I and/or II, and DNA crosslinkers.

Inhibitors of topoisomerases I and/or II include, but are not limited to, etoposide, topotecan, camptothecin, irinotecan, amsacrine, intoplicin, anthracyclines such as doxorubicin, epirubicin, daunorubicin, idarubicin and mitoxantrone. Inhibitors of Topoisomerase I and II include, but are not limited to, intoplicin.

DNA crosslinkers include, but are not limited to, cisplatin, carboplatin and oxaliplatin. In a preferred embodiment, the DNA crosslinker is cisplatin.

Anti-metabolic agents block the enzymes responsible for nucleic acid synthesis or become incorporated into DNA, which produces an incorrect genetic code and leads to apoptosis. Non-exhaustive examples thereof include, without limitation, folic acid antagonists, pyrimidine analogs, purine analogs and adenosine deaminase inhibitors, and more particularly Methotrexate, Floxuridine, Cytarabine, 6-Mercaptopurine, 6-Thioguanine, Fludarabine phosphate, Pentostatine, 5-fluorouracil, gemcitabine and capecitabine.

The DNA-damaging anti-tumoral agent can be alkylating agents including, without limitation, nitrogen mustards, ethylenimine derivatives, alkyl sulfonates, nitrosoureas, metal salts and triazenes. Non-exhaustive examples thereof include Uracil mustard, Chlormethine, Cyclophosphamide (CYTOXAN®), Ifosfamide, Melphalan, Chlorambucil, Pipobroman, Triethylenemelamine, Triethylenethiophosphoramine, Busulfan, Carmustine, Lomustine, cisplatin, carboplatin, oxaliplatin, thiotepa, Streptozocin, Dacarbazine, and Temozolomide.

The terms “kit”, “product” or “combined preparation”, as used herein, defines especially a “kit of parts” in the sense that the combination partners (a) and (b) as defined above can be dosed independently or by use of different fixed combinations with distinguished amounts of the combination partners (a) and (b), i.e. simultaneously or at different time points. The parts of the kit of parts can then, e.g., be administered simultaneously or chronologically staggered, that is at different time points and with equal or different time intervals for any part of the kit of parts. The ratio of the total amounts of the combination partner (a) to the combination partner (b) to be administered in the combined preparation can be varied. The combination partners (a) and (b) can be administered by the same route or by different routes. In a preferred embodiment, partner (b) is administered before or simultaneously partner (a). When the administration is sequential, the first partner may be for instance administered 1, 2, 3, 4, 5, 6, 12, 18 or 24 h before the second partner.

Within the context of the invention, the term treatment denotes curative, symptomatic, and preventive treatment.

Pharmaceutical compositions, kits, products and combined preparations of the invention can be used in humans with existing cancer or tumor, including at early or late stages of progression of the cancer. The pharmaceutical compositions, kits, products and combined preparations of the invention will not necessarily cure the patient who has the cancer but will delay or slow the progression or prevent further progression of the disease, ameliorating thereby the patients' condition. In particular, the pharmaceutical compositions, kits, products and combined preparations of the invention reduce the development of tumors, reduce tumor burden, produce tumor regression in a mammalian host and/or prevent metastasis occurrence and cancer relapse. In treating the cancer, the pharmaceutical composition of the invention is administered in a therapeutically effective amount.

By “effective amount” it is meant the quantity of the pharmaceutical composition of the invention which prevents, removes or reduces the deleterious effects of the treated disease in mammals, including humans. It is understood that the administered dose may be adapted by those skilled in the art according to the patient, the pathology, the mode of administration, etc. For instance, the compounds of the invention may be used at a dose of 0.01 to 500 mg/kg of body weight/day. In a particular embodiment, the pharmaceutical composition according to the invention comprises 0.01 mg to 500 mg/kg of the compound of the invention. It is understood that the administered dose may be adapted by those skilled in the art according to the patient, the pathology, the mode of administration, etc.

The treatment may be topical, transdermal, oral, rectal, sublingual, intranasal or parenteral. The pharmaceutical composition, kit, product or combined preparation is preferably administered by injection or by intravenous infusion or suitable sterile solutions, or in the form of liquid or solid doses via the alimentary canal.

The terms “cancer”, “cancerous”, or “malignant” refer to or describe the physiological condition in mammals that is typically characterized by unregulated cell growth. Examples of cancer include, for example, leukemia, lymphoma, blastoma, carcinoma and sarcoma. More particular examples of such cancers include chronic myeloid leukemia, acute lymphoblastic leukemia, Philadelphia chromosome positive acute lymphoblastic leukemia (Ph+ ALL), squamous cell carcinoma, lung cancer, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, osteosarcoma, pancreatic cancer, glioblastoma multiforme, cervical cancer, stomach cancer, bladder cancer, hepatoma, breast cancer, oesophagal cancer, colon carcinoma, and head and neck cancer, gastric cancer, germ cell tumor, pediatric sarcoma, sinonasal natural killer, multiple myeloma, acute myelogenous leukemia (AML), chronic lymphocytic leukemia, mastocytosis and any symptom associated with mastocytosis.

“Leukemia” refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease—acute or chronic; (2) the type of cell involved; myeloid (myelogenous), lymphoid (lymphogenous), or monocytic; and (3) the increase or non-increase in the number of abnormal cells in the blood—leukemic or aleukemic (subleukemic). Leukemia includes, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocyte leukemia, micromyeloblastic leukemia, monocytic leukemia, myeloblasts leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia. In certain aspects, the present invention provides treatment for chronic myeloid leukemia, acute lymphoblastic leukemia, and/or Philadelphia chromosome positive acute lymphoblastic leukemia (Ph+ ALL).

Various cancers are also encompassed by the scope of the invention, including, but not limited to, the following: carcinoma including that of the bladder (including accelerated and metastatic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, and skin (including squamous cell carcinoma); hematopoietic tumors of lymphoid lineage including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, histiocytic lymphoma, and Burketts lymphoma; hematopoietic tumors of myeloid lineage including acute and chronic myelogenous leukemias, myelodysplastic syndrome, myeloid leukemia, and promyelocytic leukemia; tumors of the central and peripheral nervous system including astrocytoma, neuroblastoma, glioma, and schwannomas; tumors of mesenchymal origin including fibrosarcoma, rhabdomyo sarcoma, and osteosarcoma; other tumors including melanoma, xenoderma pigmentosum, keratoactanthoma, seminoma, thyroid follicular cancer, and teratocarcinoma; melanoma, unresectable stage III or IV malignant melanoma, squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, retinoblastoma, gastric cancer, germ cell tumor, bone cancer, bone tumors, adult malignant fibrous histiocytoma of bone; childhood malignant fibrous histiocytoma of bone, sarcoma, pediatric sarcoma, sinonasal natural killer, neoplasms, plasma cell neoplasm; myelodysplastic syndromes; neuroblastoma; testicular germ cell tumor, intraocular melanoma, myelodysplastic syndromes; myelodysplastic/myeloproliferative diseases, synovial sarcoma. In addition, disorders include urticaria pigmentosa, mastocytosises such as diffuse cutaneous mastocytosis, solitary mastocytoma in human, as well as dog mastocytoma and some rare subtypes like bullous, erythrodermic and teleangiectatic mastocytosis, mastocytosis with an associated hematological disorder, such as a myeloproliferative or myelodysplastic syndrome, or acute leukemia, myeloproliferative disorder associated with mastocytosis, mast cell leukemia, in addition to other cancers. Other cancers are also included within the scope of disorders including, but are not limited to, the following: carcinoma, including that of the bladder, urothelial carcinoma, breast, colon, kidney, liver, lung, ovary, pancreas, stomach, cervix, thyroid, testis, particularly testicular seminomas, and skin; including squamous cell carcinoma; gastrointestinal stromal tumors (“GIST”); hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including melanoma, seminoma, teratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyo sarcoma, and osteosarcoma; and other tumors, including melanoma, xenoderma pigmentosum, keratoactanthoma, seminoma, thyroid follicular cancer, teratocarcinoma, chemotherapy refractory non-seminomatous germ-cell tumors, and Kaposi's sarcoma, and any metastasis thereof. In particular, the cancer may be selected from the group consisting of hepatoma, oesophagal cancer, breast cancer, lung cancer, neuroblastoma, ovarian cancer, colorectal cancer and osteosarcoma. Optionally, the cancer may be lung cancer, ovary cancer or osteosarcoma.

In a preferred embodiment of the present invention, the cancer is a solid tumor. The term “solid tumor” especially means breast cancer, ovarian cancer, cancer of the colon and generally the GI (gastro-intestinal) tract, cervix cancer, neuroblastoma, lung cancer, in particular small-cell lung cancer, and non-small-cell lung cancer, head and neck cancer, colorectal cancer, bladder cancer, osteosarcoma, cancer of the prostate or Kaposi's sarcoma. The present combination inhibits the growth of solid tumors, but also liquid tumors. Furthermore, a decrease of the tumor volume can be obtained. The compounds disclosed herein are also suited to prevent the metastatic spread of tumors and the growth or development of micrometastases.

Finally, the present invention relates to methods for preparing the compounds of formula (I) and (II) as disclosed above.

Accordingly, the present invention relates to a method for preparing a compound of formula (I)

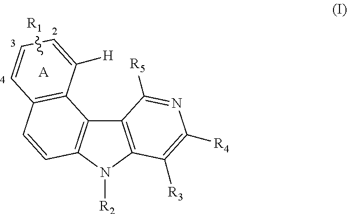

wherein R1, R2, R3, R4 and R5 are as defined above including anyone of the disclosed embodiments;

comprising

a) reacting the reagent Y—(C2-C₅)alkyl-NRₐR_{b} wherein Y is halo or hydroxy with the compound 1

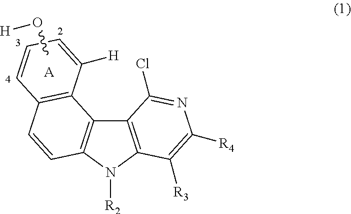

wherein Rₐ, R_{b}, R2, R3 and R4 are as defined above including anyone of the disclosed embodiments;

and

b) reacting the compound obtained at step a) with alkali (C₁-C₄)alkoxide, thereby obtaining the compound of formula (I).

In a particular embodiment, alkali (C₁-C₄)alkoxide is a sodium (C₁-C₄)alkoxide.

The reactions of step a) and step b) are carried out in the appropriate conditions.

In particular for step a), the conditions are for instance the followings: i) for the Williamson condensation using halogeno-(C₂-C₅)alkyl-NRₐR_{b} as reagent: the reaction was realized in phase-transfer conditions (BuOH-water) and in the presence of alkali hydroxide as a base and ii) for the Mitsunobu reaction using HO—(C₂-C₅)alkyl-NRₐR_{b} and dialkyl azodicarboxylate as reagents: the condensation was realized in standard conditions.

Preferably, Halogeno-(C₂-C₅)alkyl-NRₐR_{b} is chloro-(C₂-C₅)alkyl-NRₐR_{b} and dialkyl azodicarboxylate is diisopropyl azodicarboxylate.

In particular for step b), the conditions are for instance the followings: the alkoxylation of the chloro derivative was realized by heating in sealed tube at high temperature in the presence of alkali alkoxide.

Preferably, alkali alkoxide is sodium methoxide.

The present invention also relates to a method for preparing a compound of formula (II)

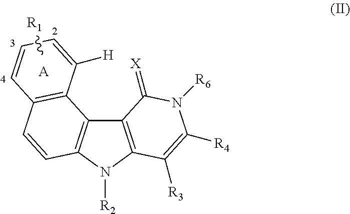

wherein R1, R2, R3, R4, R6 and X are as defined above including anyone of the disclosed embodiments;

comprising

a) reacting the reagent Y—(C₂-C₅)alkyl-NRₐR_{b} wherein Y is halo or hydroxy with the compound 1

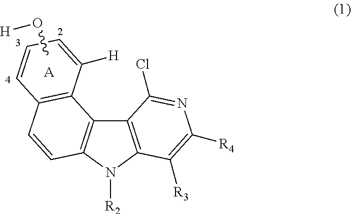

wherein R2, R3, R4 and R6 are as defined above including anyone of the disclosed embodiments;

and

b) reacting the compound obtained at step a) either i) with carboxylic acid anhydride, or ii) with alkali carboxylate in carboxylic acid, thereby obtaining the compound of formula (II).

The reactions of step a) and step b) are carried out in the appropriate conditions.

In particular for step a), the conditions are for instance the followings: i) for the Williamson condensation using halogeno-(C₂-C₅)alkyl-NRₐR_{b} as reagent: the reaction was realized in phase-transfer conditions (BuOH-water) and in the presence of alkali hydroxide as a base and ii) for the Mitsunobu reaction using HO—(C₂-C₅)alkyl-NRₐR_{b} and dialkyl azodicarboxylate as reagents: the condensation was realized in standard conditions.

Preferably, Halogeno-(C₂-C₅)alkyl-NRₐR_{b} is chloro-(C₂-C₅)alkyl-NRₐR_{b} and dialkyl azodicarboxylate is diisopropyl azodicarboxylate.

In particular for step b), the conditions are for instance the followings: the transformation of the chloro derivative into lactame compound was realized either by heating in sealed tube at high temperature in carboxylic acid anhydride, or by heating in carboxylic acid in the presence of alkali carboxylate.

Preferably, carboxylic acid anhydride is acetic anhydride, carboxylic acid is acetic acid and alkali carboxylate is sodium acetate.

Alternatively, the compound of formula (II) may also be prepared from the intermediate product obtained at step a) of the method for preparing the compound of formula (I) by heating the intermediate product obtained at step a) of the method for preparing the compound of formula (I). In particular, the conditions are for instance the followings: the dealkylation of the alkoxy derivative was realized by heating with aqueous hydracide in acetic acid.

Preferably, hydracid is hydrochloride acid.

Further aspects and advantages of the present invention will be disclosed in the following experimental section, which should be regarded as illustrative and not limiting the scope of the present application. A number of references are cited in the present specification; each of these cited references is incorporated herein by reference.

### EXAMPLES

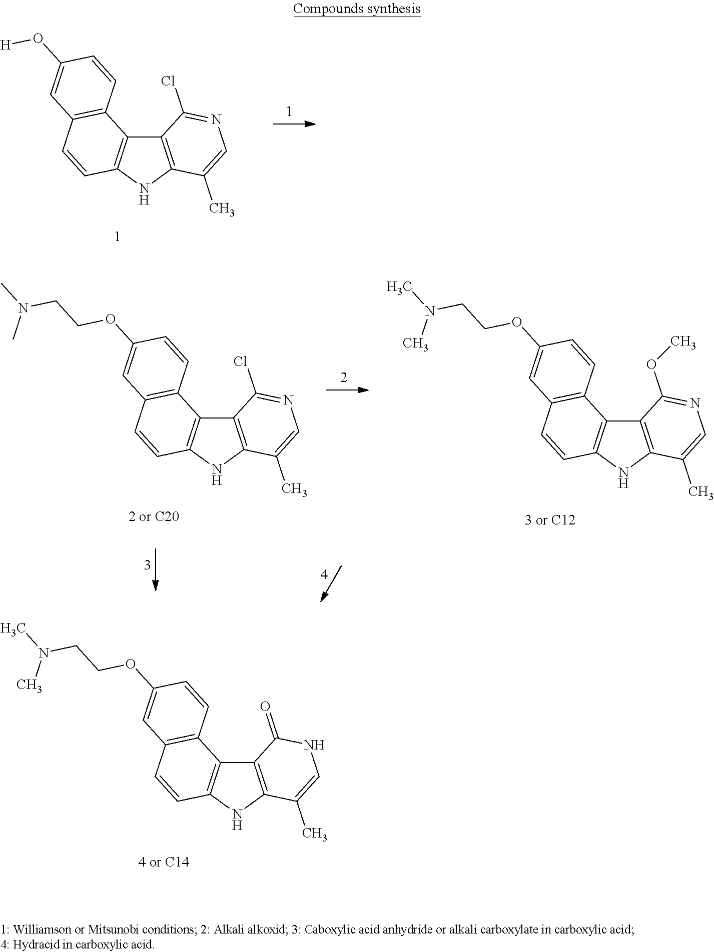 [SMILES: Cc1cnc(Cl)c2c1[nH]c1ccc3cc(O)ccc3c12 ; Cc1cnc(Cl)c2c1[nH]c1ccc3cc(OCCN(C)C)ccc3c12 ; COc1ncc(C)c2[nH]c3ccc4cc(OCCN(C)C)ccc4c3c12 ; Cc1c[nH]c(=O)c2c1[nH]c1ccc3cc(OCCN(C)C)ccc3c12]

### 11-Chloro-3-(2-N,N-dimethylaminoethoxy)-8-methyl-7H-benzo[e]pyrido(4,3-b)indole (2 or C20)

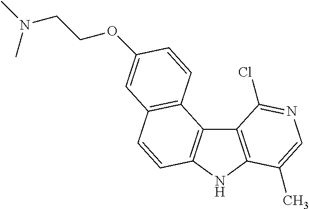 [SMILES: Cc1cnc(Cl)c2c1[nH]c1ccc3cc(OCCN(C)C)ccc3c12]

Method 1:

A mixture of 11-chloro-3-hydroxy-8-methyl-7H-benzo[e]pyrido(4,3-b)indole (400 mg, 1.4 mmol) (prepared according to Bioconjugate Chem. (2003), 14, 120-135), n-butanol (40 mL) and water (24 mL) was stirred during 30 min and a solution of NaOH (300 mg) in water (5 mL) was then added. After stirring during 15 min, 2-chloro-N,N-dimethylethanamine hydrochloride (240 mg, 1.7 mmol) was added and the mixture was heated under reflux for 1 h. The reaction mixture was cooled and organic layer was separated. The aqueous layer was extracted by AcOEt and the organic layers were combined, washed with brine, dried over MgSO₄ and evaporated under vacuum. The residue was purified by flash chromatography (neutral alumina, gradient of ethanol (0 to 2%) in dichloromethane) to give the expected compound as beige solid (220 mg, 44%). ¹H NMR (300 MHz, CDCl₃) δ (ppm): 9.76 (d, 1H), 8.79 (br s, 1H), 8.10 (s, 1H), 7.85 (d, 1H), 7.62 (d, 1H), 7.39 (dd, 1H), 7.35 (d, 1H), 4.24 (t, 2H), 2.83 (t, 2H), 2.56 (s, 3H), 2.40 (s, 6H). Microanalyses, calculated for C₂₀H₂₀ClN₃O.0.7H₂O: C, 65.64; H, 5.85; N, 11.48. found: C, 65.29; H, 5.49; N, 11.31.

Method 2:

Under N2 atmosphere, diisopropyl azodicarboxylate (280 mg, 1.4 mmol) was added to a solution of triphenylphosphine (430 mg, 1.4 mmol) in dry THF (10 mL). This mixture was added to a solution of 11-chloro-3-hydroxy-8-methyl-7H-benzo[e]pyrido(4,3-b)indole (200 mg, 0.7 mmol) (prepared according to Bioconjugate Chem. (2003), 14, 120-135) and 2-(dimethylamino)ethanol (75 mg, 0.8 mmol) in dry THF (20 mL). The final mixture was stirred for 24 h at room temperature. The solvent was evaporated. The crude residue was purified by column chromatography as described above to give the expected compound which is identical to that obtained by method 1.

### 11-Methoxy-3-(2-N,N-dimethylaminoethoxy)-8-methyl-7H-benzo[e]pyrido(4,3-b)indole (3 or C12)

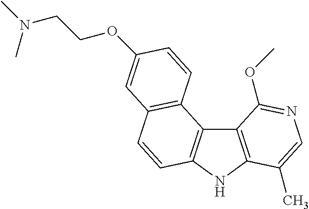 [SMILES: COc1ncc(C)c2[nH]c3ccc4cc(OCCN(C)C)ccc4c3c12]

In a 25 mL sealed tube, a mixture of 11-chloro-3-(2-N,N-dimethylaminoethoxy)-8-methyl-7H-benzo[e]pyrido(4,3-b)indole (80 mg, 0.22 mmol) and 30% sodium methoxide in methanol solution (12 mL) was heated in an oil bath at 130° C. for 48 h. The reaction mixture was cooled, poured into water (30 mL) and extracted by CH₂Cl₂. The organic layer was washed with brine, dried over MgSO₄ and evaporated under vacuum. The residue was purified by flash chromatography (neutral alumina, gradient of AcOEt (0 to 20%) in CH₂Cl₂) to give the free base of methoxy compound (75 mg, 94%). ¹H NMR (300 MHz, CDCl₃) δ (ppm): 9.64 (d, 1H), 8.82 (br s, 1H), 7.85 (s, 1H), 7.73 (d, 1H), 7.58 (d, 1H), 7.35 (dd, 1H), 7.30 (d, 1H), 4.27-4.22 (m, 5H), 2.83 (t, 2H), 2.46 (s, 3H), 2.39 (s, 6H).

Formation of the maleate salt: A solution of this free base (35 mg) in hot acetone (5 mL) was poured into a solution of maleic acid (33 mg) in hot acetone (2 mL). The precipitate was collected by filtration, washed with acetone and dried in a dessicator under vacuum affording the maleat salt (40 mg). Microanalyses, calculated for C₂₁H₂₃N₃O₂.2C₄H₄O₄.H₂O: C, 58.10; H, 5.50; N, 7.01. found: C, 57.96; H, 5.44; N, 7.12. MS 350.2 [M+1].

### 3-(2-N,N-Dimethylaminoethoxy)-8-methyl-7H,10H-benzo[e]pyrido(4,3-b)indol-11-one (4 or C14)

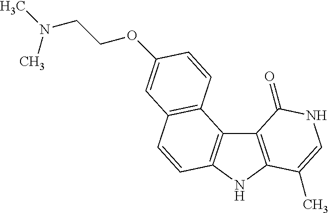 [SMILES: Cc1c[nH]c(=O)c2c1[nH]c1ccc3cc(OCCN(C)C)ccc3c12]

Method 1:

A mixture of 11-methoxy-3-(2-N,N-dimethylaminoethoxy)-8-methyl-7H-benzo[e]pyrido(4,3-b)indole (40 mg, 0.11 mmol), N HCl (5 mL) and AcOH (2 mL) was heated under reflux for 18 h. The volatiles were evaporated under vacuum followed by a co-evaporation with toluene. Water (10 mL) was added and the medium was rendered basic by addition of 28% ammonium hydroxide (2 mL). The aqueous layer was extracted by AcOEt and the organic layer was washed with brine, dried over MgSO₄ and evaporated under vacuum. The residue was purified by flash chromatography (neutral alumina, gradient of ethanol (0 to 10%) in dichloromethane) to give: (i) recovered starting material (22 mg) and (ii) the expected compound as beige solid (12 mg, 69%). ¹H NMR (300 MHz, CDCl₃) δ (ppm): 10.28, (d, 1H), 8.89 (br s, 1H), 8.60 (br s, 1H), 7.70 (d, 1H), 7.56 (d, 1H), 7.37 (dd, 1H), 7.29 (d, 1H), 7.02 (s, 1H), 4.24 (t, 2H), 2.84 (t, 2H), 2.40 (s, 6H), 2.35 (s, 3H). MS 336.3 [M+1].

Method 2:

In a 25 mL sealed tube, a mixture of 11-chloro-3-(2-N,N-dimethylaminoethoxy)-8-methyl-7H-benzo[e]pyrido(4,3-b)indole (230 mg, 0.65 mmol) and acetic anhydride (20 mL) was heated in an oil bath at 150° C. for 20 h. The volatile was evaporated under vacuum followed by a co-evaporation with toluene. Toluene (6 mL) was added and the precipitate was collected by filtration, washed with toluene. The obtained residue was purified by flash chromatography (neutral alumina, gradient of ethanol (0 to 10%) in CH₂Cl₂) to give the expected compound (160 mg, 73%) which is identical to that obtained by method 1.

Method 3:

A mixture of 11-chloro-3-(2-N,N-dimethylaminoethoxy)-8-methyl-7H-benzo[e]pyrido(4,3-b)indole (290 mg, 0.82 mmol), sodium acetate (172 mg, 2.1 mmol) and AcOH (16 mL) was heated under reflux for 18 h. The volatile was evaporated under vacuum followed by a co-evaporation with toluene. Water (100 mL) was added and the medium was rendered basic by addition of 2 N sodium hydroxide. The aqueous layer was extracted by AcOEt and EtOH, dried over MgSO₄ and evaporated under vacuum to give the expected compound (274 mg, 100%) which is identical to that obtained by methods 1. Microanalyses, calculated for C₂₀H₂₁N₃O₂.1.8H₂O: C, 65.32; H, 6.69; N, 11.43. found: C, 65.36; H, 6.28; N, 11.17.

Formation of the maleate salt: A solution of this free base (120 mg) in boiling absolute ethanol (10 mL) was poured into a solution of maleic acid (50 mg) in hot absolute ethanol (2 mL). The homogenous solution obtained was evaporated under vacuum and the residue was triturated with acetone giving a solid which was collected by filtration, washed with acetone and dried in a dessicator affording the maleate salt (160 mg). Microanalyses, calculated for C₂₀H₂₁N₃O₂.C₄H₄O₄.0.5H₂O: C, 62.61; H, 5.65; N, 9.31. found: C, 62.51; H, 5.80; N, 9.42.

### 11-Chloro-3-(3-N,N-dimethylaminopropoxy)-8-methyl-7H-benzo[e]pyrido(4,3-b)indole

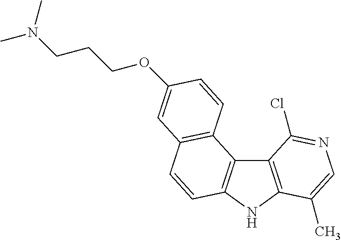 [SMILES: Cc1cnc(Cl)c2c1[nH]c1ccc3cc(OCCCN(C)C)ccc3c12]

A mixture of 11-chloro-3-hydroxy-8-methyl-7H-benzo(e)pyrido(4,3-b)indole (300 mg, 1.1 mmol) (prepared according to Bioconjugate Chem. (2003), 14, 120-135), n-butanol (30 mL) and water (18 mL) was stirred during 30 min and a solution of NaOH (200 mg) in water (3 mL) was then added. After stirring during 15 min, (CH₃)₂N(CH₂)₃Cl, HCl (200 mg, 1.3 mmol) was added and the mixture was heated under reflux for 1 h. The reaction mixture was cooled and organic layer was separated. The aqueous layer was extracted by AcOEt and the organic layers were combined, washed with brine, dried over MgSO₄ and evaporated under vacuum. The residue was purified by flash chromatography (neutral alumina, gradient of ethanol (0 to 2%) in dichloromethane) to give the expected compound 11-chloro-3-(3-N,N-dimethylaminopropoxy)-8-methyl-7H-benzo(e)pyrido(4,3-b)indole as beige solid (190 mg, 48%). Microanalyses, calculated for C₂₁H₂₂ClN₃O.0.5H₂O: C, 66.92; H, 6.15; N, 11.15. found: C, 66.73; H, 5.92; N, 11.27.

### 3-(3-N,N-Dimethylaminopropoxy)-8-methyl-7H,10H-benzo[e]pyrido(4,3-b)indol-11-one

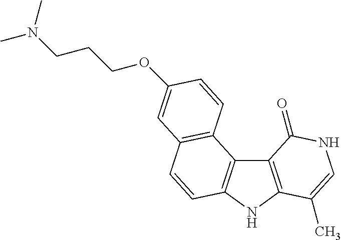 [SMILES: Cc1c[nH]c(=O)c2c1[nH]c1ccc3cc(OCCCN(C)C)ccc3c12]

The method 2 described above for the synthesis of compound 3-(2-N,N-dimethylaminoethoxy)-8-methyl-7H,10H-benzo(e)pyrido(4,3-b)indol-11-one is applied, starting from 11-chloro-3-(3-N,N-dimethylamino prop oxy)-8-methyl-7H-benzo(e)pyrido(4,3-b)indole, prepared as describe above, to give the title compound.

### 11-Chloro-3-(2-N,N-diethylaminoethoxy)-8-methyl-7H-benzo[e]pyrido(4,3-b)indole

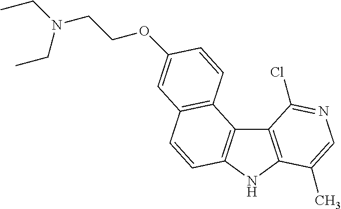 [SMILES: CCN(CC)CCOc1ccc2c(ccc3[nH]c4c(C)cnc(Cl)c4c32)c1]

The protocol described above for the synthesis of compound 11-chloro-3-(2-N,N-dimethylaminoethoxy)-8-methyl-7H-benzo[e]pyrido(4,3-b)indole is applied, starting from 11-chloro-3-hydroxy-8-methyl-7H-benzo[e]pyrido(4,3-b)indole and using 2-chloro-N,N-diethylethanamine hydrochloride in place of 2-chloro-N,N-dimethylethanamine hydrochloride, to give the title compound.

### 3-(2-N,N-Diethylaminoethoxy)-8-methyl-7H,10H-benzo[e]pyrido(4,3-b)indol-11-one

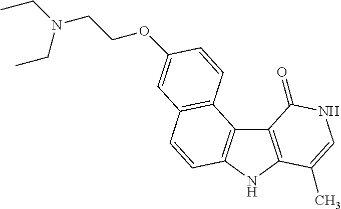 [SMILES: CCN(CC)CCOc1ccc2c(ccc3[nH]c4c(C)c[nH]c(=O)c4c32)c1]

The method 2 described above for the synthesis of compound 3-(2-N,N-dimethylaminoethoxy)-8-methyl-7H,10H-benzo[e]pyrido(4,3-b)indol-11-one is applied, starting from 11-chloro-3-(2-N,N-diethylaminoethoxy)-8-methyl-7H-benzo[e]pyrido(4,3-b)indole, prepared as describe above, to give the title compound.

### 11-Chloro-3-(2-(morpholin-4-yl)ethoxy)-8-methyl-7H-benzo[e]pyrido(4,3-b)indole

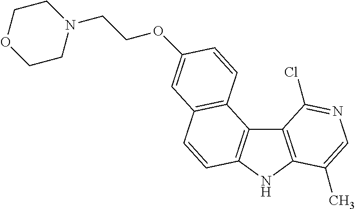 [SMILES: Cc1cnc(Cl)c2c1[nH]c1ccc3cc(OCCN4CCOCC4)ccc3c12]

The protocol described above for the synthesis of compound 11-chloro-3-(2-N,N-dimethylaminoethyloxy)-8-methyl-7H-benzo[e]pyrido(4,3-b)indole was applied, starting from 11-chloro-3-hydroxy-8-methyl-7H-benzo(e)pyrido(4,3-b)indole and using 4-(2-chloroethyl)morpholine hydrochloride in place of 2-chloro-N,N-dimethylethanamine hydrochloride, to give the title compound. ¹H NMR (300 MHz, DMSO-d₆) δ (ppm): 12.94 (br s, 1H), 9.62 (d, 1H), 8.06 (d, 1H), 7.99 (d 1H), 7.80 (d, 1H), 7.59 (d, 1H), 7.35 (dd, 1H), 4.25 (t, 2H), 3.64-3.58 (m, 4H), 2.78 (t, 2H), 2.56 (s, 3H), 2.54-2.49 (m. overlapped by DMSO signals). Microanalyses, calculated for C₂₂H₂₀ClN₃O₂.0.5H₂O: C, 65.34; H, 5.69; N, 10.39. found: C, 65.75; H, 5.73; N, 10.37. MS 394.1 & 396.1 [M−1].

### 3-(2-(Morpholin-4-yl)ethoxy)-8-methyl-7H,10H-benzo[e]pyrido(4,3-b)indol-11-one (CH 1707 or C07)

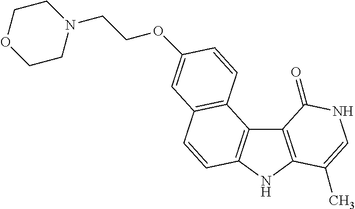 [SMILES: Cc1c[nH]c(=O)c2c1[nH]c1ccc3cc(OCCN4CCOCC4)ccc3c12]

The method 2 or 3 described above for the synthesis of compound 3-(2-N,N-dimethylaminoethoxy)-8-methyl-7H,10H-benzo[e]pyrido(4,3-b)indol-11-one was applied, starting from 11-chloro-3-(2-(morpholin-4-yl)ethoxy)-8-methyl-7H-benzo(e)pyrido(4,3-b)indole, prepared as describe above, to give the title compound. ¹H NMR (300 MHz, DMSO-d₆) δ (ppm): 12.03 (br s, 1H), 10.94 & 10.92 (2 s, 1H), 10.26 (d, 1H), 7.75-7.64 (m, 2H), 7.42 (d, 1H), 7.18 (dd, 1H), 7.07 (d, 1H), 4.21 (t, 2H), 3.66-3.56 (m, 4H), 2.77 (t, 2H), 2.57-2.46 (m. overlapped by DMSO signals). Microanalyses, calculated for C₂₂H₂₃N₃O₃.1.2H₂O: C, 66.23; H, 6.37; N, 10.54. found: C, 66.28; H, 6.55; N, 10.10. MS 400.1 [M+Na].

Formation of the maleate salt: A solution of this free base (150 mg) in boiling absolute ethanol (10 mL) was poured into a solution of maleic acid (55 mg) in hot absolute ethanol (4 mL). The homogenous solution obtained was evaporated under vacuum and the residue was triturated with acetone giving a solid which was collected by filtration, washed with acetone and dried in a dessicator affording the maleate salt (160 mg). Microanalyses, calculated for C₂₂H₂₃N₃O₃.C₄H₄O₄.0.4H₂O: C, 62.37; H, 5.56; N, 8.40. found: C, 62.66; H, 6.00; N, 7.95.

### DL 11-Chloro-3-(3-N,N-dimethylamino-2-methylpropoxy)-8-methyl-7H-benzo[e]pyrido(4,3-b)indole

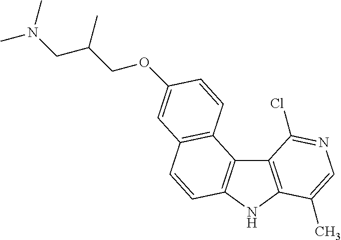 [SMILES: Cc1cnc(Cl)c2c1[nH]c1ccc3cc(OCC(C)CN(C)C)ccc3c12]

The protocol described above for the synthesis of compound 11-chloro-3-(2-N,N-dimethylaminoethyloxy)-8-methyl-7H-benzo[e]pyrido(4,3-b)indole is applied, starting from 11-chloro-3-hydroxy-8-methyl-7H-benzo(e)pyrido(4,3-b)indole and using DL 3-chloro-2-methyl-N,N-dimethylpropan-1-amine hydrochloride in place of 2-chloro-N,N-dimethylethanamine hydrochloride, to give the title compound.

### DL 3-(3-N,N-dimethylamino-2-methylpropoxy)-8-methyl-7H-10H-benzo[e]pyrido(4,3-b)indol-11-one

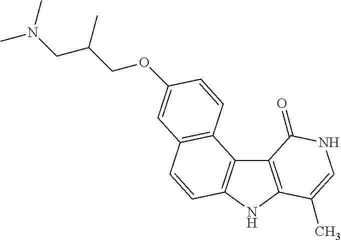 [SMILES: Cc1c[nH]c(=O)c2c1[nH]c1ccc3cc(OCC(C)CN(C)C)ccc3c12]

The method 2 described above for the synthesis of compound 3-(2-N,N-dimethylaminoethoxy)-8-methyl-7H,10H-benzo[e]pyrido(4,3-b)indol-11-one is applied, starting from DL 11-Chloro-3-(3-N,N-dimethylamino-2-methylpropoxy)-8-methyl-7H-benzo[e]pyrido(4,3-b)indole, prepared as describe above, to give the title compound.

### 11-Chloro-3-(3-(piperidin-1-yl)propoxy)-8-methyl-7H-benzo[e]pyrido(4,3-b)indole

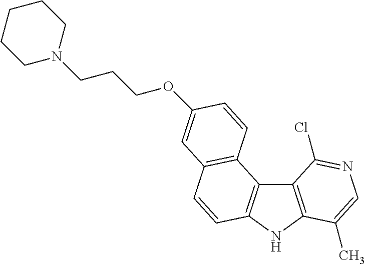 [SMILES: Cc1cnc(Cl)c2c1[nH]c1ccc3cc(OCCCN4CCCCC4)ccc3c12]

The protocol described above for the synthesis of compound 11-chloro-3-(2-N,N-dimethylaminoethyloxy)-8-methyl-7H-benzo[e]pyrido(4,3-b)indole is applied, starting from 11-chloro-3-hydroxy-8-methyl-7H-benzo(e)pyrido(4,3-b)indole and using 1-(3-chloropropyl)piperidine hydrochloride in place of 2-chloro-N,N-dimethylethanamine hydrochloride, to give the title compound.

### 3-(3-(Piperidin-1-yl)propoxy)-8-methyl-7H,10H-benzo[e]pyrido(4,3-b)indol-11-one

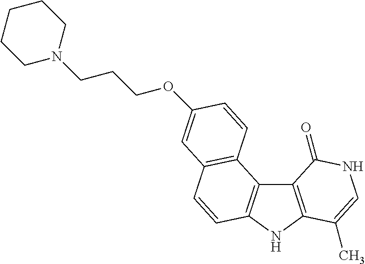 [SMILES: Cc1c[nH]c(=O)c2c1[nH]c1ccc3cc(OCCCN4CCCCC4)ccc3c12]

The method 2 described above for the synthesis of compound 3-(2-N,N-dimethylaminoethoxy)-8-methyl-7H,10H-benzo[e]pyrido(4,3-b)indol-11-one is applied, starting from 11-chloro-3-(3-(piperidin-1-yl)prop oxy)-8-methyl-7H-benzo(e)pyrido(4,3-b)indole, prepared as describe above, to give the title compound.

### 11-Chloro-3-(2-N,N-dimethylaminoethoxy)-8-ethyl-7H-benzo[e]pyrido(4,3-b)indole

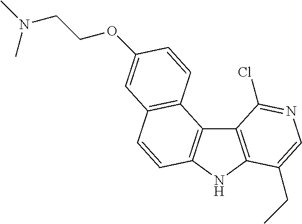 [SMILES: CCc1cnc(Cl)c2c1[nH]c1ccc3cc(OCCN(C)C)ccc3c12]

Step 1: In a 50 mL sealed tube, a mixture of 11-chloro-8-ethyl-3-methoxy-7H-benzo[e]pyrido(4,3-b)indole (600 mg, 1.9 mmol) (prepared according to Anti-Cancer Drug Design (1992), 7, 235-251), benzyltriethylammonium chloride (2.80 g, 12 mmol) and 37% hydrochloric acid (45 mL) was heated in an oil bath at 140° C. for 24 h. The reaction mixture was then evaporated under vacuum, then water (10 mL) was added. The medium was rendered basic by addition of 28% ammonium hydroxide (2 mL), and the resulting solid was collected by filtration, then washed with water and dried using a vacuum desiccator. The intermediate 11-chloro-8-ethyl-3-hydroxy-7H-benzo(e)pyrido(4,3-b)indole 450 mg (78% yield) was obtained as brown powder. Microanalyses, calculated for C₁₇H₁₃ClN₂O0.25H₂O: C, 67.78; H, 4.52; N, 9.30. found: C, 67.90; H, 4.62; N, 9.56. MS: 296.1/298.1 (M+H).

Step 2: The protocol described above for the synthesis of compound 11-chloro-3-(2-N,N-dimethylaminoethyloxy)-8-methyl-7H-benzo[e]pyrido(4,3-b)indole was applied, starting from 11-chloro-3-hydroxy-8-ethyl-7H-benzo[e]pyrido(4,3-b)indole and using 2-chloro-N,N-dimethylethanamine hydrochloride, to give the title compound. ¹H NMR (300 MHz, CDCl₃) δ (ppm): 9.76 (d, 1H), 8.73 (br s, 1H), 8.13 (s, 1H), 7.86 (d, 1H), 7.62 (d, 1H), 7.43-7.34 (m, 2H), 4.25 (t, 2H), 2.97 (q, 2H), 2.84 (t, 2H), 2.40 (s, 6H), 1.46 (t, 3H). MS: 368.2 & 370.2 (M+H).

### 3-(2-N,N-dimethylaminoethoxy)-8-ethyl-7H,11H-benzo[e]pyrido(4,3-b)indol-11-one (C48)

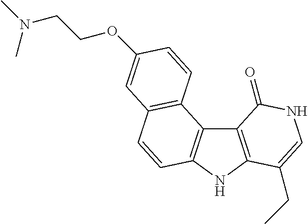 [SMILES: CCc1c[nH]c(=O)c2c1[nH]c1ccc3cc(OCCN(C)C)ccc3c12]

The method 2 or 3 described above for the synthesis of compound 3-(2-N,N-dimethylaminoethoxy)-8-methyl-7H,10H-benzo[e]pyrido(4,3-b)indol-11-one was applied, starting from 11-chloro-3-(2-N,N-dimethylaminoethoxy)-8-ethyl-7H-benzo[e]pyrido(4,3-b)indole, prepared as describe above, to give the title compound. ¹H NMR (300 MHz, DMSO-d₆) δ (ppm): 12.02 (s, 1H), 10.98 & 10.96 (2 s, 1H), 10.27 (d, 1H), 7.74-7.67 (m, 2H), 7.40 (d, 1H), 7.18 (dd, 1H), 7.05 (d, 1H), 4.17 (t, 2H), 2.77-2.67 (m, 4H), 2.26 (s, 6H), 1.27 (t, 2H). Microanalyses, calculated for C₂₁H₂₃N₃O₂.H₂O: C, 68.66; H, 6.81; N, 11.44. found: C, 68.96; H, 6.78; N, 11.36. MS 350.1 [M+H].

Formation of the maleate salt: A solution of this free base (120 mg) in boiling absolute ethanol (12 mL) was poured into a solution of maleic acid (62 mg) in hot absolute ethanol (2 mL). The homogenous solution obtained was evaporated under vacuum and the residue was triturated with acetone giving a solid which was collected by filtration, washed with acetone and dried in a dessicator affording the maleate salt (147 mg). Microanalyses, calculated for C₂₁H₂₃N₃O₂.C₄H₄O₄.0.25H₂O: C, 63.89; H, 5.85; N, 8.94. found: C, 63.41; H, 6.28; N, 8.76.

### 11-Chloro-3-(2-(piperidin-1-yl)ethoxy)-8-methyl-7H-benzo[e]pyrido(4,3-b)indole (CH 1709 or C709)

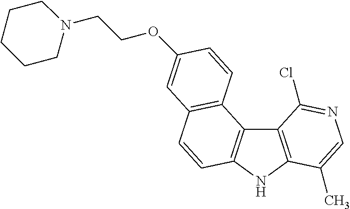 [SMILES: Cc1cnc(Cl)c2c1[nH]c1ccc3cc(OCCN4CCCCC4)ccc3c12]

The protocol described above for the synthesis of compound 11-chloro-3-(2-N,N-dimethylaminoethyloxy)-8-methyl-7H-benzo[e]pyrido(4,3-b)indole was applied, starting from 11-chloro-3-hydroxy-8-methyl-7H-benzo[e]pyrido(4,3-b)indole and using N-(2-chloroethyl)piperidine hydrochloride in place of 2-chloro-N,N-dimethylethanamine hydrochloride, to give the title compound. ¹H NMR (300 MHz, DMSO-d₆) δ (ppm): 12.48 (br s, 1H), 9.61 (d, 1H), 8.06 (s, 1H), 7.99 (d 1H), 7.79 (d, 1H), 7.58 (d, 1H), 7.34 (dd, 1H), 4.22 (t, 2H), 2.74 (t, 2H), 2.56 (s, 3H), 2.51-2.45 (m. overlapped by DMSO signals), 1.56-1.47 (m, 4H), 1.45-1.30 (m, 2H). Microanalyses, calculated for C₂₃H₂₄ClN₃O₂: C, 70.13; H, 6.14; N, 10.67. found: C, 69.63; H, 6.38; N, 10.44. MS 394.1 & 396.1 [M+1].

### 3-(2-(Piperidin-1-yl)ethoxy)-8-methyl-7H,10H-benzo[e]pyrido(4,3-b)indol-11-one (CH 1710 or C710)

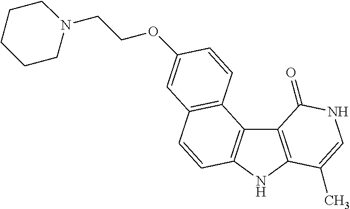 [SMILES: Cc1c[nH]c(=O)c2c1[nH]c1ccc3cc(OCCN4CCCCC4)ccc3c12]

The method 2 or 3 described above for the synthesis of compound 3-(2-N,N-dimethylaminoethoxy)-8-methyl-7H,10H-benzo[e]pyrido(4,3-b)indol-11-one was applied, starting from 11-chloro-3-(2-(piperidin-1-yl)ethoxy)-8-methyl-7H-benzo(e)pyrido(4,3-b)indole, prepared as describe above, to give the title compound. ¹H NMR (300 MHz, DMSO-d₆) δ (ppm): 12.03 (br s, 1H), 10.94 & 10.92 (2 br s, 1H), 10.26 (d, 1H), 7.73-7.63 (m, 2H), 7.41 (d, 1H), 7.17 (dd, 1H), 7.08 (d, 1H), 4.18 (t, 2H), 2.73 (t, 2H), 2.56-2.43 (m, overlapped by DMSO signals), 2.29 (s, 3H), 1.57-1.48 (m, 4H), 1.44-1.35 (m, 2H). Microanalyses, calculated for C₂₃H₂₅N₃O₂H₂O: C, 70.22; H, 6.87; N, 10.68. found: C, 70.16; H, 6.92; N, 10.42. MS 376.2 [M+1].

Formation of the maleate salt: A solution of this free base (160 mg) in boiling absolute ethanol (14 mL) was poured into a solution of maleic acid (60 mg) in hot absolute ethanol (4 mL). The homogenous solution obtained was evaporated under vacuum and the residue was triturated with acetone giving a solid which was collected by filtration, washed with acetone and dried in a dessicator affording the maleate salt (180 mg). Microanalyses, calculated for C₂₃H₂₅N₃O₂.C₄H₄O₄.0.5H₂O: C, 64.80; H, 6.00. found: C, 64.41; H, 6.37.

### 3-(2-N,N-dimethylaminoethyloxy)-8-methyl-7H-benzo[e]pyrido(4,3-b)indole (C21 or CH21)

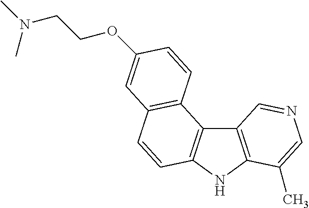 [SMILES: Cc1cncc2c1[nH]c1ccc3cc(OCCN(C)C)ccc3c12]

Palladium on carbon (10%, 150 mg) was added to a solution of 11-chloro-3-(2-N,N-dimethylaminoethyloxy)-8-methyl-7H-benzo[e]pyrido(4,3-b)indole (300 mg, 0.84 mmol) in absolute ethanol (30 mL). At atmospheric pressure, hydrogen was introduced and the mixture was stirred during 18 h. The catalyst was then filtered off, washed with hot ethanol and the solvent was removed under reduced pressure. The residue was purified by flash chromatography (neutral alumina, gradient of ethanol (0 to 2%) in dichloromethane) to give the expected compound 3-(2-N,N-dimethylaminoethyloxy)-8-methyl-7H-benzo[e]pyrido(4,3-b)indole as beige solid (190 mg, 71%). ¹H NMR (300 MHz, DMSO-d₆) δ (ppm): 12.39 (s, 1H), 9.73 (s, 1H), 8.77 (d, 1H), 8.37 (s, 1H), 7.99 (d, 1H), 7.86 (d, 1H), 7.68 (d, 1H), 7.47 (dd, 1H), 4.48 (t, 2H), 2.79 (s, 6H), 2.65 (s, 3H), 2.53 (m, 2H). Microanalyses, calculated for C₂₀H₂₁N₃O.0.25H₂O: C, 74.18; H, 6.64; N, 12.98. found: C, 74.21; H, 6.67; N, 12.71.

### 3-(3-N,N-dimethylaminopropoxy)-8-methyl-7H-benzo[e]pyrido(4,3-b)indole (C23)

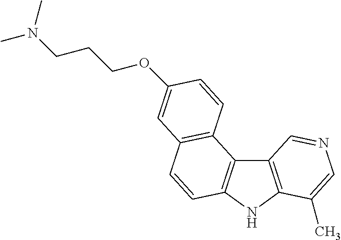 [SMILES: Cc1cncc2c1[nH]c1ccc3cc(OCCCN(C)C)ccc3c12]

Palladium on carbon (10%, 70 mg) was added to a solution of 11-chloro-3-(3-N,N-dimethylaminopropoxy)-8-methyl-7H-benzo[e]pyrido(4,3-b)indole (130 mg, 0.35 mmol) in absolute ethanol (20 mL). At atmospheric pressure, hydrogen was introduced and the mixture was stirred during 18 h. The catalyst was then filtered off, washed with hot ethanol and the solvent was removed under reduced pressure. The residue was purified by flash chromatography (neutral alumina, gradient of ethanol (0 to 2%) in dichloromethane) to give the expected compound as beige solid (84 mg, 71%). ¹H NMR (300 MHz, DMSO-d₆) δ (ppm): 12.07 (s, 1H), 9.64 (s, 1H), 8.70 (d, 1H), 8.31 (s, 1H), 7.95 (d, 1H), 7.80 (d, 1H), 7.59 (d, 1H), 7.39 (dd, 1H), 4.20 (t, 2H), 2.62 (s, 3H), 2.47 (t, 2H), 2.22 (s, 6H), 1.98 (m, 2H). Microanalyses, calculated for C₂₁H₂₃N₃O.0.25H₂O: C, 74.64; H, 7.01; N, 12.43. found: C, 74.77; H, 6.99; N, 12.45.

### 11-Chloro-3-(2-methoxyethoxy)-8-methyl-7H-benzo[e]pyrido(4,3-b)indole (CH1708)

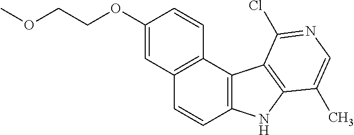

The protocol described above for the synthesis of compound 11-chloro-3-(2-N,N-dimethylaminoethyloxy)-8-methyl-7H-benzo[e]pyrido(4,3-b)indole was applied, starting from 11-chloro-3-hydroxy-8-methyl-7H-benzo(e)pyrido(4,3-b)indole and using 1-chloro-2-methoxyethane in place of 2-chloro-N,N-dimethylethanamine hydrochloride to give the title compound. Notice that in this case the mixture was heated at 70° C. for a period of 4 h. ¹H NMR (300 MHz, CDCl₃) δ (ppm): 9.78 (d, 1H), 8.70 (br s, 1H), 8.11 (s, 1H), 7.86 (d, 1H), 7.63 (d, 1H), 7.41 (dd, 1H), 7.36 (d, 1H), 4.30 (t, 2H), 3.86 (t, 2H), 3.51 (s, 3H), 2.57 (s, 3H). MS 341.0 & 343.0 [M+1].

### 3-(2-Methoxyethoxy)-8-methyl-7H,10H-benzo[e]pyrido(4,3-b)indol-11-one (CH1711)

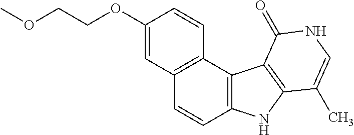

The method 3 described above for the synthesis of compound 3-(2-N,N-dimethylaminoethoxy)-8-methyl-7H,10H-benzo[e]pyrido(4,3-b)indol-11-one was applied, starting from 11-chloro-3-(2-methoxyethoxy)-8-methyl-7H-benzo(e)pyrido(4,3-b)indole, prepared as describe above, to give the title compound. ¹H NMR (300 MHz, DMSO-d₆) δ (ppm): 12.09 (s, 1H), 11.01 & 10.99 (2 s, 1H), 10.33 (d, 1H), 7.80-7.70 (m, 2H), 7.46 (d, 1H), 7.26 (dd, 1H), 7.15 (d, 1H), 4.28 (t, 2H), 3.80 (t, 2H), 3.41 (s, 3H), 3.35 (s, 3H). MS 323.2 [M+1].

### Characterization of the effects of Amino-substituted-alkyloxy-benzo[e]pyrido[4,3-b]indole family

### C14 or 3-(2-Dimethylaminoethyloxy)-8-methyl-7H,10H-benzo[e]pyrido(4,3-b)indol-11-one

1) Anti-Proliferating Activity

TABLE 1
Efficiency of C14 towards cell line viability. Lowest IC50 are underlined
in bolt. Data obtained with C1 and CH21 are added for comparison.

| | Cell | IC 50 for C14 | IC50 for C1 | IC50 for CH21 |
|---|---|---|---|---|
| Origin | line | In nM | In nM | In nM |
| Lung | H322 | Sup 4 000 | ND | 800 |
| Lung | H358 | 470 | 145 | 450 |
| Leukemia | HL60 | Sup 2 000 | 350 | Sup 2 000 |
| Cervix | HeLa | 110 | 960 | 350 |
| Liver cells | SkHep1 | 2 000 | ND | 450 |
| Osteosarcoma | U2OS | 720 | Sup 1 000 | 1 100 |
| Glioblastoma | U373 | 3100 | Sup 1 000 | 360 |

C14 exhibits antiproliferative activity towards varying cell lines (viability IC50 110 nM in HeLa cells and 470 nM in H358 cells) (see Table 1). These results do not parallel activities obtained with two other benzopyridoindoles sharing some structural characteristics (C1 and CH21). C1 inhibits efficiently HL60 proliferation whereas C14 and CH21 are poor inhibitors. CH21 prevents U373 cell proliferation whereas C 14 and C1 are inefficient. Therefore these molecules may have different specificities.

Antiproliferative assays with HeLa cells have been performed with the preferred compounds and compared to the C1 and C2. The average of three independent experiments is shown. IC50 obtained with C1 and C2 were previously reported in Hoang et al, 2009, and are indicated for comparison. The lateral chain of each molecule is recalled, knowing that all the molecules have an oxo group at position 11. IC50 are indicated, i.e. the concentration that induced 50% death.

| Molecule | R1 | R3 | IC50 (in nM) |
|---|---|---|---|
| C2 | O—Me | Me | 1160 ± 150 |
| C1 | O—Me | Et | 960 ± 140 |
| C14 | O—(CH2)₂—N(CH3)₂ | Me | 170 ± 82 |
| C48 | O—(CH2)₂—N(CH3)₂ | Et | 53 ± 3 |
| C07 | 2-(Morpholin-4-yl)ethoxy | Me | 107 ± 12 |
| C14M | O—(CH2)₂—N(CH3)₂ | Me | 114 ± 82 |
| C48M | O—(CH2)₂—N(CH3)₂ | Et | 63 ± 17 |
| C07M | 2-(Morpholin-4-yl)ethoxy | Me | 287 ± 32 |

The dimethylaminoethoxy group increases dramatically the antiproliferative efficiency (i.e., compare C2 and C14, C1 and C48). However, similar effect has been observed with the morpholin-4-ylethoxy group.

2) Kinase Profiling of Compound C14

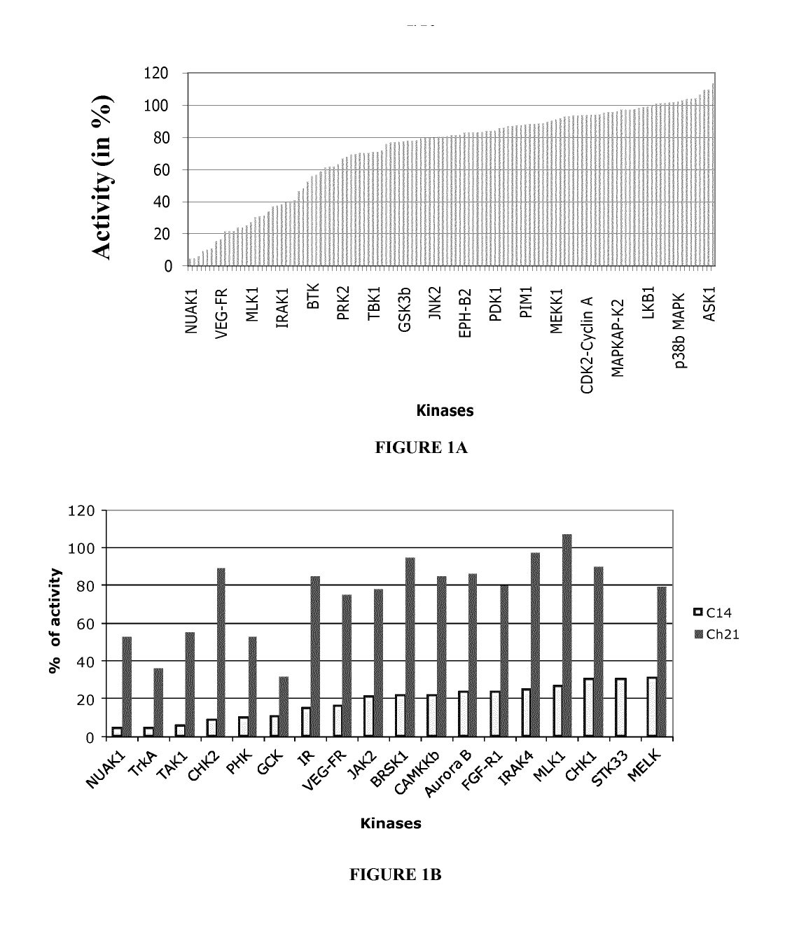

The specificity of C14 was tested, in vitro, against 121 kinases representative of the kinome. Assays run in duplicate, at the concentration of 1 μM, were performed by MRC (Dundee, UK). Results are represented in FIG. 1. The main targets are highlighted in FIG. 1B.

TABLE 2
Main kinases inhibited by C14 at 1 microMolar in vitro.

| | Percent of | |
|---|---|---|
| Kinases | remaining activity | Deviation |
| NUAK1 | 4 | 4 |
| TrkA | 5 | 3 |
| Tak1 | 6 | 1 |
| Chk2 | 9 | 1 |
| Phk | 10 | 1 |
| Gck | 11 | 1 |
| IR | 15 | 2 |
| VEGFR | 16 | 6 |
| Jak2 | 21 | 5 |
| Brsk1 | 22 | 4 |
| CAMKKb | 22 | 4 |
| Aurora B | 24 | 3 |
| FGFR1 | 24 | 0 |
| IRAK4 | 25 | 3 |
| MLK1 | 27 | 4 |
| CHK1 | 31 | 1 |
| STK33 | 31 | 10 |
| MELK | 31 | 9 |

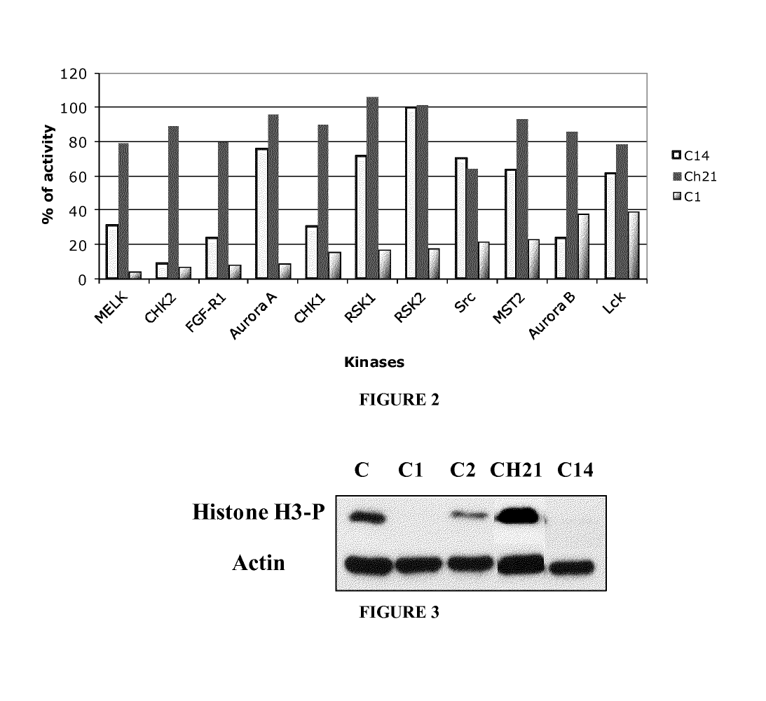

In addition to kinases also targeted by CH21 (Nuak1, TrkA, Tak1 and Gck), C14 inhibits mitotic kinases (Aurora B and MELK) as well as checkpoint kinases 1 and 2. Aurora A is only partly inhibited by C14 (76% of inhibition, at 1 μM). In fact, C14 shares few specificities with C1 but not all. For example, it is a poor Aurora A inhibitor whereas C1 targets in similar way Aurora A and B (Hoang et al, 2009). One may expect thus different impairment of mitosis by both molecules C1 and C14. The specificities of the different available inhibitors are compared in Table 3 and FIG. 2.

TABLE 3

| Compound | Molecule | Known Targets (%) |
|---|---|---|
| C1 | 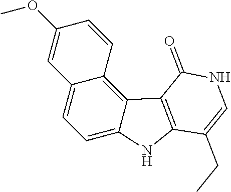 [SMILES: CCc1c[nH]c(=O)c2c1[nH]c1ccc3cc(OC)ccc3c12] | Aurora A Aurora B Chk2 FLT3 Melk |
| C2 | 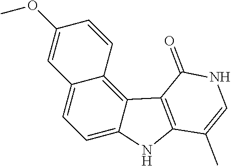 [SMILES: COc1ccc2c(ccc3[nH]c4c(C)c[nH]c(=O)c4c32)c1] | Aurora A Aurora B Chk1 Chk2 Flt3 |
| C21 or CH21 | 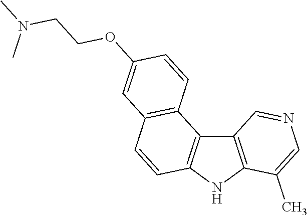 [SMILES: Cc1cncc2c1[nH]c1ccc3cc(OCCN(C)C)ccc3c12] | NUAK1 TrkA Tak1 Gck Flt3 |
| C14 | 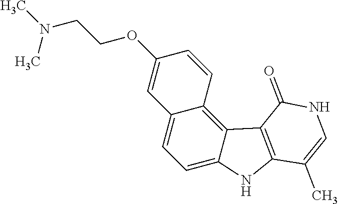 [SMILES: Cc1c[nH]c(=O)c2c1[nH]c1ccc3cc(OCCN(C)C)ccc3c12] | Aurora B NUAK1 TrkA Tak1 Gck Chk1 Chk2 |

comparison of the kinase inhibition specificities in the Benzo[e]pyridoindole family. The lists are not exhaustive but only include the list of the kinases presenting the highest inhibitions when the data is available.

In addition, the percentage of remaining activity of recombinant kinases Aurora kinases A and B and Checkpoint kinases 1 and 2 at the concentration of 1 μM of C1, C2 and C14 have been determined. The results are shown in the below table.

| Kinases | C1 | C2 | C14 |
|---|---|---|---|
| Aurora A | 7% | 11% | 76% |
| Aurora B | 19% | 24% | 24% |
| Chk1 | 23% | 12% | 31% |
| Chk2 | 6% | 3% | 9% |

In view of these data, it becomes apparent that only C14, compared to C1 and C2, showed a specificity to Aurora B versus Aurora A. Therefore, the dimethylaminoethoxy group surprisingly modifies the specificity of the compounds.

3) C14 Targets Aurora Kinases in Cellulo

The main substrate of Aurora B is histone H3. Aurora B phosphorylates Histone H3 on Ser 10 during mitosis. U2OS mitotic extracts treated by the above compounds were analyzed by western blotting. As shown in FIG. 3, C1 and C14 inhibit efficiently Aurora B, in cellulo, whereas CH21 has no effect and C2 a mild inhibitory activity.

4) Structure/Function Study

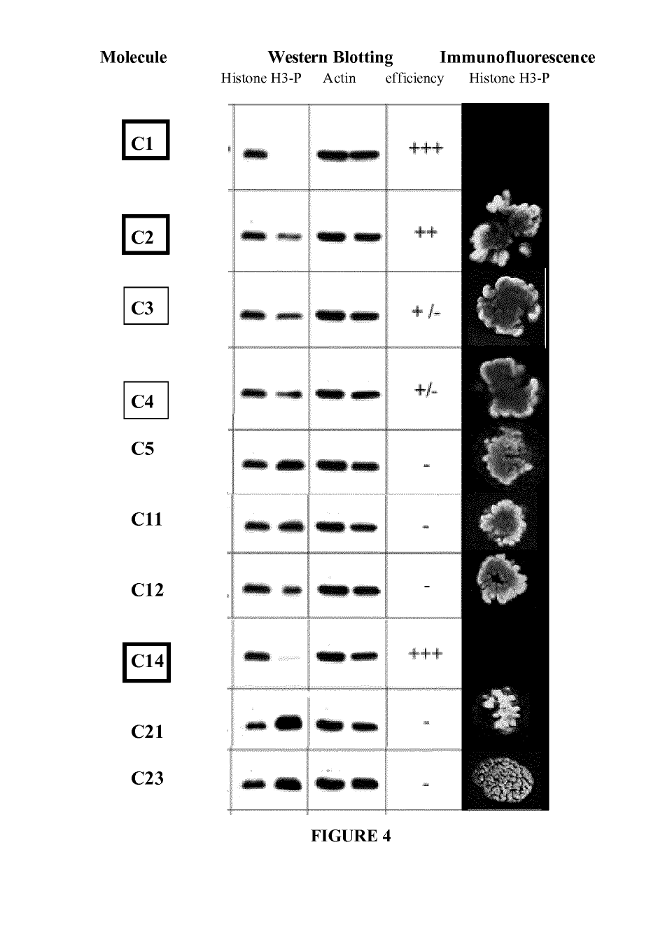

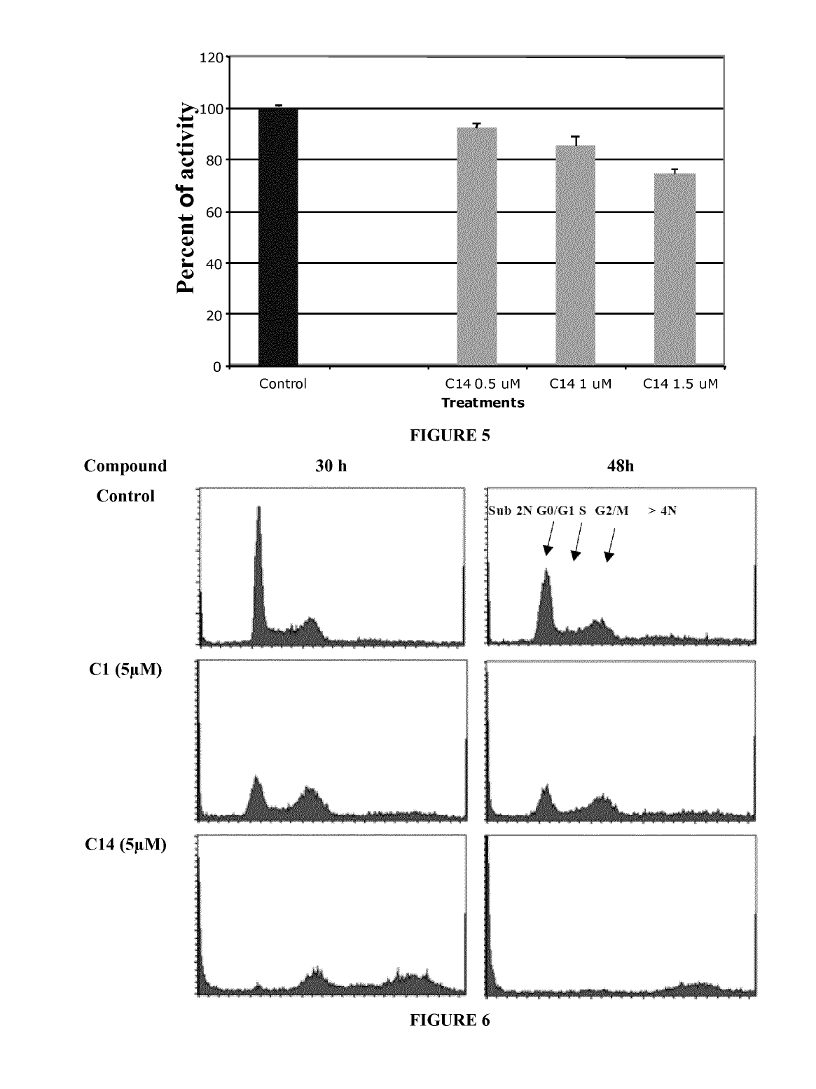

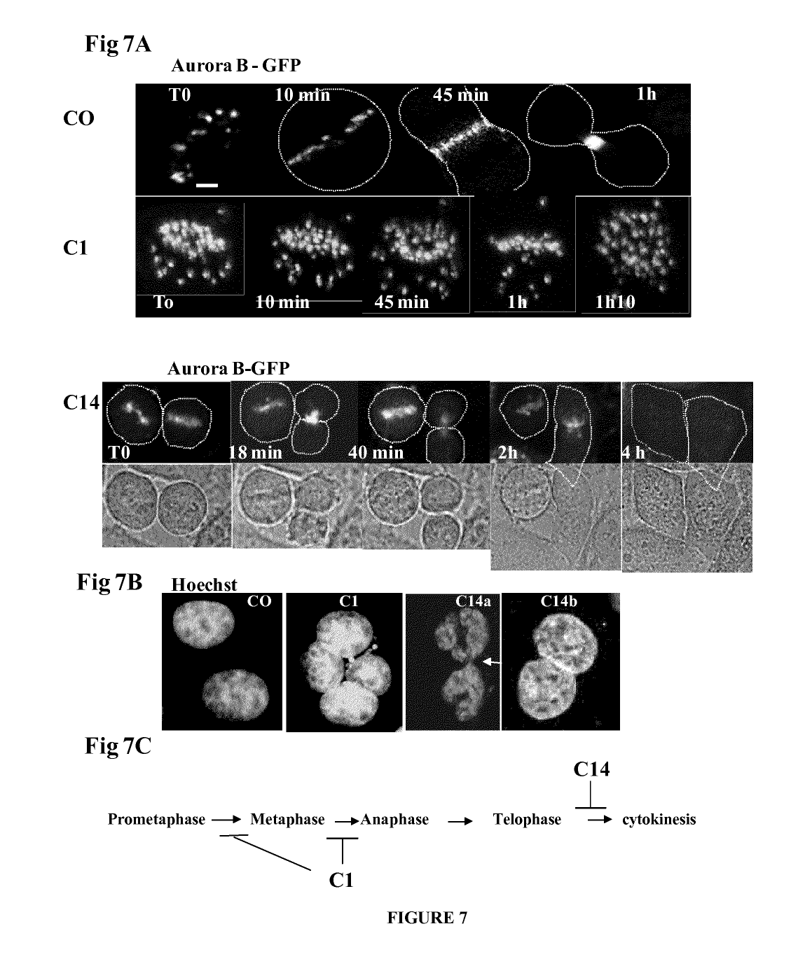

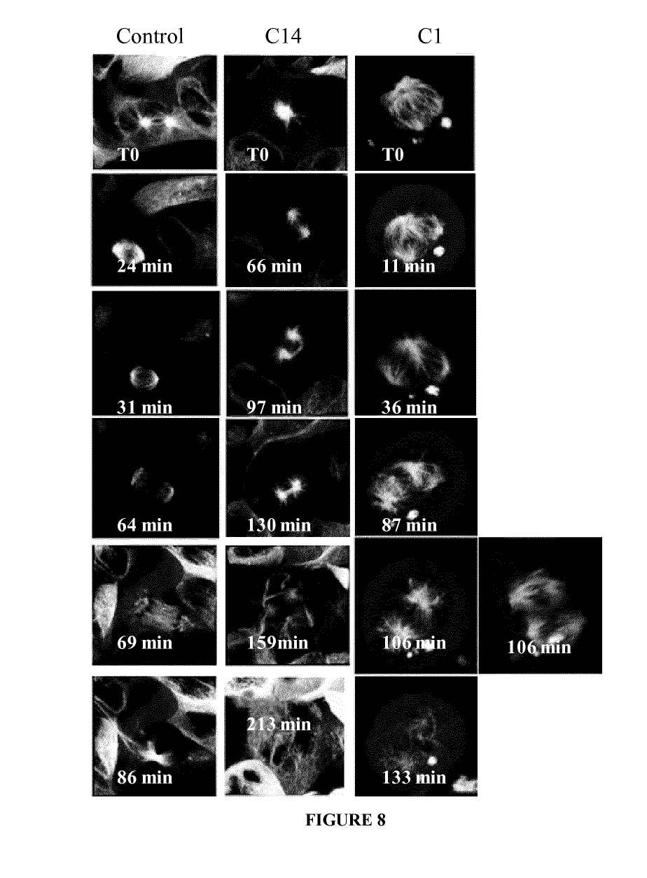

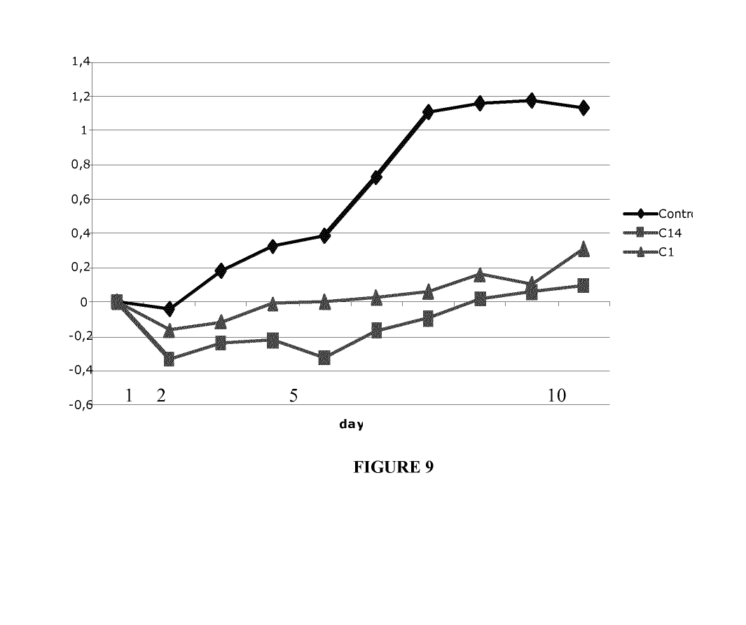

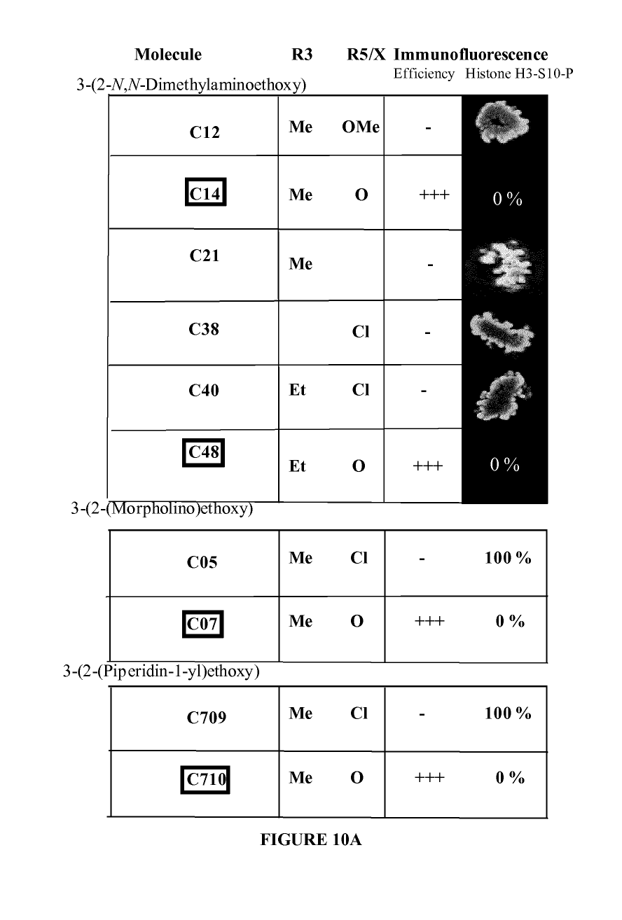

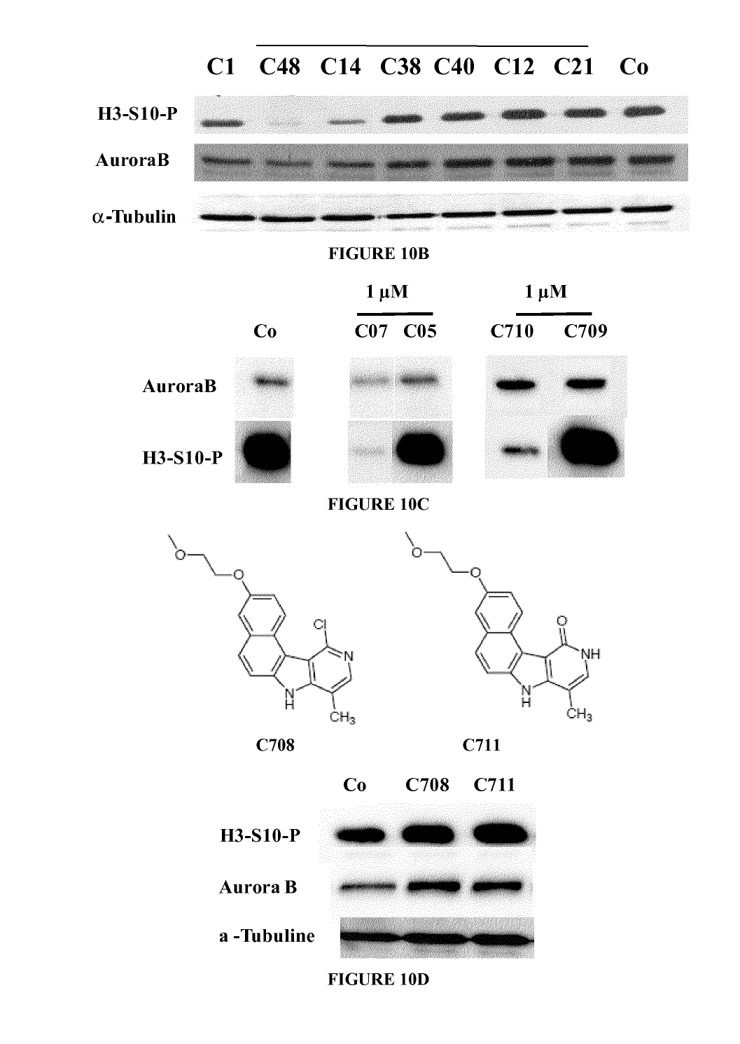

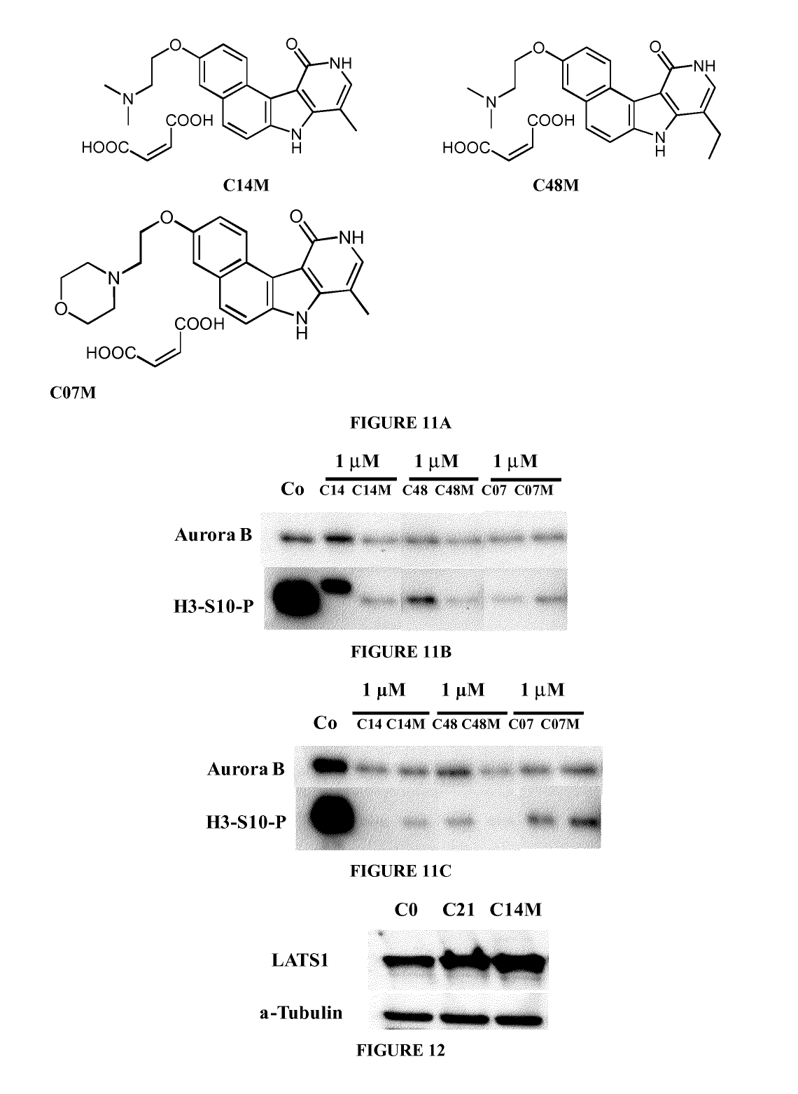

Cells were incubated with the different molecules and Aurora B inhibition is estimated by western blotting and immunofluorescent experiments. Histone H3 phosphorylated on Ser10 is used as a marker of Aurora B kinase activity and actin as an internal control. Results are represented in FIGS. 4 and 10 and Table 3.

Compounds C1-C5 have been disclosed in Hoang et al, 2009 in FIG. 1 as following:

| Compound | R₅ | R₂ |
|---|---|---|
| 1 | Et | 3-OMe |
| 2 | Me | 3-OMe |
| 3 | Me | H |
| 4 | H | 3-OMe |
| 5 | Me | 4-OMe |

with

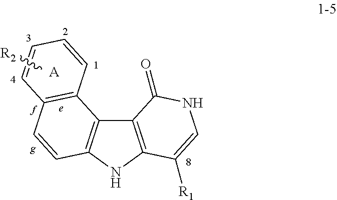

Compound C11 has the following structure and it was described in J. Med. Chem. (1990), 33, 1519-1528:

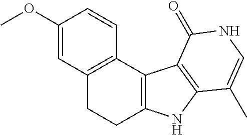

TABLE 4
Structure/function analysis in the benzo[e]pyridoindole family.

| | | | | Aurora B |
|---|---|---|---|---|
| Compound | R1 | R3 | X in formula I or R5 in formula 2 | inhibition |
| C1 | 3-O—Me | Et | X = O | +++ |
| C2 | 3-O—Me | Me | X = O | ++ |
| C3 | 3-H | Me | X = O | +/− |
| C4 | 3-O—Me | H | X = O | +/− |
| C5 | 4-O—Me | Me | X = O | − |
| C11 | 3-O—Me | Me | X = O; 5,6-dihydro | − |
| C12 | 3-O—(CH₂)₂—N(CH₃)₂ | Me | R5 = O—Me | − |
| C14 | 3-O—(CH₂)₂—N(CH₃)₂ | Me | X = O | +++ |
| C21 or CH21 | 3-O—(CH₂)₂—N(CH₃)₂ | Me | R5 = H | − |
| C23 | 3-O—(CH₂)₂—N(CH₃)₂ | Me | R5 = H | − |
| C38 | 3-O—(CH₂)₂—N(CH₃)₂ | H | R5 = Cl | − |
| C40 | 3-O—(CH₂)₂—N(CH₃)₂ | Et | R5 = Cl | − |
| C48 | 3-O—(CH₂)₂—N(CH₃)₂ | Et | X = O | +++ |
| C05 | 2-(morpholin-4-yl)ethoxy | Me | R5 = Cl | − |
| C07 | 2-(morpholin-4-yl)ethoxy | Me | X = O | +++ |
| C709 | 2-(piperidin-1-yl)ethoxy | Me | R5 = Cl | − |
| C710 | 2-(piperidin-1-yl)ethoxy | Me | X = O | +++ |
| C708 | 2-methoxyethoxy | Me | R5 = Cl | − |
| C711 | 2-methoxyethoxy | Me | X = O | − |

Comparison of C2 and C14 reveals that the large chain in position 3 of the benzo cycle A highly increases the inhibition of Aurora B kinase and the selectivity. An ethyl in position 8 (R3 in formula I or II) may be valuable for a better targeting. The position 11 (R5 in formula I) is also essential to Aurora B kinase inhibition since the activity is loosed when the oxo group is turned to a hydrogen (compare C14 and C21 also named CH21) or to a chloride (compare C40 and C48 or C05 and C07 or C709 and C710).

Maleic salts corresponding to Aurora B kinase inhibitors C14, C48 and C07, respectively called C 14M, C48M and C07M, have been synthesized and tested (FIG. 11). The Aurora B kinase inhibition is maintained for the maleate salts.

5) Effect of C14 on Quiescent Cells

H358 cells were deprived in serum for 2 days and then incubated with the compound, for 96 h, still in the absence of serum. At the concentration (0.5 μM) that induced death of more than 50% of cycling cells, 90% of the cells are still alive 96 hours post-treatment (FIG. 5). C14 is thus not deleterious for quiescent cells.

6) Effect of C14 on Cell Cycle Progression

HeLa cells were incubated in the presence of either C1 (0.5 μM) or C14 (0.5 μM) or DMSO (Te) and the reparation of the cells in the different phases is analyzed by FACS. Analyses were performed upon 30 h and 48h of treatment and histograms are reported in FIG. 6 and data are shown in Table 5.

TABLE 5
repartition of the cells upon C1 and C14 treatments. Major modifications
between C1 and C14 are indicated by bold numbers.

| | | | S | G2/M | |
|---|---|---|---|---|---|
| Treatment | <2N in % | G0/G1 in % | in % | in % | >4N in % |
| Control | 4 | 45 | 16 | 20 | 15 |
| C1 (0.5 μM, 30 h) | 6 | 24 | 13 | 28 | 26 |
| C14 (0.5 μM, 30 h) | 19 | 3 | 4 | 18 | 56 |
| C1 (0.5 μM, 48 h) | 6 | 18 | 8 | 22 | 41 |
| C14 (0.5 μM, 48 h) | 22 | 2 | 2 | 4 | 56 |

C14 prevents efficiently cell cycling (only 7% at 30 h and 5% at 48 h are in G0/G1 and S). In the presence of C14, a massive apoptosis and polyploidisation are noted only 30 hours after its addition. Under the same conditions, C1 affects cell cycle only after one cell generation and leads to polyploidy only after 48 h of treatment. Therefore, C14 prevents efficiently HeLa proliferation and induces cell death. Moreover, cells are still cycling in the presence of C1 (48% of the cells after 48 h of treatment). These results fit perfectly with the IC50 observed for both compounds in HeLa cells. These data confirmed that C14 and C1 affects differently HeLa cell signalling.

7) Time-Lapses of HeLa Cells Expressing Aurora B-GFP

HeLa cells expressing aurora B-GFP were treated by either C14 (0.5 μM) or C1 (0.5 μM) or DMSO (control; Co). Mitotic cells were continuously imaged under controlled conditions (37° C. and 5% CO2). The localisation of aurora B-GFP is represented and the elapsed time indicated in the three conditions (FIG. 7A). Control cells divided normally; Aurora B presented on the centromeres (FIG. 7A, CO, To and 10 min) was then transferred on microtubules (FIG. 7A, CO 45 min) and finally concentrated in the mid body (FIG. 7A, CO, 1 h). In such condition, the daughter cells exhibited two well-separated nuclei (FIG. 7B, CO). In the presence of C1 (0.5 μM), Aurora B concentrated on centromere, but chromosome failed to align on the metaphasic plate (FIG. 7A, C1; 1 h) and finally they misaligned (FIG. 7A, C1; 1 h 10). These cells escaped from mitosis without anaphase and gave rise to micronuclei (FIG. 7B, C1). In the presence of C14 (0.5 μM), chromosome aligned normally (FIG. 7B, C14 To) and aurora B-GFP was found accumulated in the mid-zone (FIG. 7B, C14, 18 min, 40 min and 2 h). Finally, these cells spread again without division (FIG. 7B, C14 4 h). Actually, as shown in (FIG. 7C, C14a), the two nuclei are connected (see arrow) and cannot be individualized (FIG. 7 C, C14b).

In other words both C1 and C14 impaired mitosis; C1 prevented anaphase ongoing whereas C14 affected cytokinesis.

8) Time-Lapses of Hek Cells Expressing Tubulin-GFP

Mitotic Hek cells stably expressing tubulin-GFP were continuously imaged and representative of either control or treated cells (C14 and C1, 0.5 μM) are shown (FIG. 8). Elapse times are indicated on each photo. In the control as well as in the presence of C1 the mitotic spindle is organized (24 min and To respectively) whereas in the presence of C14, the two poles are only connected by few microtubules (97 min and 130 min). Finally, in the presence of C14, cells escape from mitosis and cytoplasmic microtubules are observed (159 min and 212 min). Note that a duplication of the spindle occurs upon C1 treatment (106 min)

9) Effect of C14 on Spheroids

In order to get some insight in C14 effects towards organized cells, the inventors have followed H358 spheroid expansion (FIG. 9). Organized 3-dimension spheroids were treated by either C14 (0.5 μM) or C1 (0.5 μM) or DMSO (control). C14 affected significantly 3D-H358 cell growth and its effects lasted at least 5 days. Finally, at day 10, C14 treated spheroids were found smaller than their counterparts treated by C1. C14 was thus found efficient to prevent cell growth even when cells mimicked tissue organization.

10) C14 Targets AMPK-Related Kinase 5 (Nuak1), in Cellulo

The profiling revealed that AMPK-related kinase 5 (Nuakl) is the most affected kinase in the panel. The efficiency of the 3-amino-ethoxy compounds to inhibit Nuak1 was determined in vitro.

| Compounds | IC50 (Nuak1) in nM |
|---|---|
| C14 | 1.4 |
| C48 | 5.7 |
| C07 | 1.6 |

Therefore the possibility of inhibiting Nuakl, in cellulo, was evaluated through the stabilization of the Large Antigen Tumour Suppressor LATS1. As expected the inhibition of Nuak1 induced a large increase of LATS1 protein as reported in FIG. 12.

11) Conclusion

C14 is a new, water soluble, antiproliferating compound. C14 is a narrow kinase inhibitor that targets mitotic kinases like aurora B and, AMPK-related kinases like Nuak1 and MELK. It impairs cytokinesis and may therefore prevent mitosis ongoing especially in stem cancer cells expressing MELK (Nakano et al, 2008). Due to the targeting of MELK, C14 may also be proposed for cancer stem cells targeting (neuroblastoma, colorectal cancers, lung and breast, . . . ). C14 may have great interest towards deregulated myc tumours since it was reported that their proliferation depends upon AMPK-related kinase 5 (Nuak1), the main target of C14 (Liu et al, 2012).

C14 targets Gck, a MAP4K (Ippeita et al., 2001) and Trks enzymes (Thiele et al., 2009) and may have some inhibitory potentiality in MAP cascade signalling, in inflammatory process and pain sensation (Jian et al., 2009; Watson et al. 2008). Gck inhibitors are of interest for anti-cancer and anti-inflammatory therapeutic intervention (Ippeita et al., 2001; Jian et al., 2007). Trk or Tropomysin related kinases are described as oncogene and play a role in chronic inflammatory pain (Harel et al., 2010; Thiele et al., 2009). TRK are receptors for neurotrophins; Neurotrophins being a family of secreted growth factors that are broadly implicated as causative in pain-associated with a variety of human diseases. In terms of cancer, Trk receptors are involved in resistant tumours like hepatoma or oesophageal cancers. An interesting application is described in (Albanese et al, 2010). Actually, it is proposed that Trk inhibitors may be used to prevent cell proliferation meanwhile decreasing pain induced by bone metastatic invasion for example.

C14 exhibited interesting cell proliferation inhibition without major toxicity on quiescent cells. C14 is more efficient than previously described benzo[e]pyridoindole C1 and exhibits a different pattern of kinase inhibition, in particular Aurora B selectivity. Moreover, C1 and C14 impaired differently mitosis; C1 blocking the metaphase to anaphase transition while C14 preventing cytokinesis. To note, C14 also targets Chk1, this kinase being involved in DNA repair as well as mitosis completion (Peddibhotla et al., 2009). The targeting of checkpoint kinases suggests the benefit of the combined used with DNA damaging agents.

These features allowed proposing C14 as a new anti-proliferation compound useful for targeted polypharmacology (Apsel et al, 2008). The major advantage of multispecific kinase inhibitors is to increase efficiency with a unique drug therefore decreasing side effects (Harrison, 2010).

Finally, taking into account the identification of Aurora-like homologs in various organisms (fungi, plants, Trypanosoma brucei, . . . ), broader applications of aurora inhibitors may be considered (Jetton et al., 2009). These molecules may be proposed as an anti-parasite compounds.

In addition to C14, other compounds of interest have been identified. The compounds of interest always bear an Oxo group in position 11, a group dimethylaminoethoxy or 2-(morpholin-4-yl)ethoxy group or 2-(piperidinyl)ethoxy group in position 3 and a methyl or ethyl in position 8. These compounds have an increased antiproliferative activity, show specificity for Aurora B over Aurora A, and are inhibitors of Chk-1 and Chk-2. In particular, the compounds C14, C48, C07 and C710, and the maleate salts thereof, are promising.

10) Materials and Methods.

Cell Culture and Cell Viability Test.

H358, H322 (lung cancer cells), U373 (glioblastomes) and HeLa (ovarian cancer) were grown in DMEM (1.5 g/L glucose) whereas HL60 (human myeloid cell line) cells were in RPMI 1640. U2OS (a human osteosarcoma cell line) cells were cultured in McCoy's Medium 5 A and liver cells (SK Hep 1) in DMEM (4 g/L glucose, (Gibco-Invitrogen). Media (Gibco-Invitrogen), were supplemented with 10% heat-inactivated foetal bovine serum (Gibco-Invitrogen), L-glutamine (2 mM), penicillin (100 UI/ml) and streptomycin (100 μg/ml).

Cell proliferation assays were conducted in 96 well culture plates. Assays were run in triplicate. Cell viability was estimated upon 72 h of treatment with varying concentrations of compound by addition of CellTiter 96Queous one Solution Reagent (Promega) directly to culture wells under conditions defined by the manufacturer.

Cells were drawn to quiescence by serum withdrawn. After two days of serum deprivations cells were incubated with different concentrations of compounds for 96 hours, serum being still omitted. The viability is determined as described above.

Western Blot.

Cells were treated by compounds, harvested and lyzed in 9M urea then supplemented with Laemmli sample buffer. Cell extracts were subjected to SDS-PAGE and transferred to nitrocellulose filter (GE Healthcare). After blocking with 5% non-fat milk in PBS for at least 1 hour, the membranes were incubated with the primary antibodies. The following antibodies were used: phospho-Histone H3 Ser10 (1:2000, Upstate Biotechnology), anti LAST1 (Cell signalling), anti Aurora B (epitomics) and anti alpha-tubulin (Sigma) Anti-rabbit horseradish peroxidase (1:5000, GE Healthcare) was used as secondary antibodies. Bands were visualized by ECL technique (Amersham Bioscience).

Cell Cycle Analysis.

Cells were incubated in either the presence or the absence of compounds. For determination of cell cycle profile, cells were fixed by ice-cold 70% ethanol for 1 h and then, incubated with propidium iodide solution (50 μg/ml PI in the presence of 0.2 mg/ml RNAse) for 15 minutes at 37° C. DNA content was measured using the FACS flow cytometer equipped with CellQuest Pro software (Becton Dickinson, San Diego, Calif.).

Immunofluorescence.

Cells were seeded on glass coverslips and treated by compounds (1 μM) overnight. After treatment, cells were fixed in 4% formaldhehyde at 37° C. for 15 minutes, then permeabilized for 5 minutes with Triton X-100 in PBS. After blocking in 0.05 mg/ml BSA for 30 minutes, the primary antibody was added (anti phospho-histone H3 Ser10 (1/2000, Upstate). After 30 min of incubation, unbound antibodies were removed by washing with PBS, 0.2% Tween-20 and specific staining was revealed with Hylite Fluor™ 546- or 647- or 488-conjugated secondary antibodies (Anaspec). DNA was visualized with 0.1 mM Hoechst 33342 (Sigma). Images were collected with a ZEISS 510 Laser Scanning Confocal microscope with a 63× immersion oil objective. Slices of 0.5 micron are shown.

Time-Lapse Experiments.

HeLa cells stably expressing aurora B-GFP were continuously imaged under cultured conditions (37° C. and 5% CO2). Cells were either incubated in the presence of C14 (0.5 μM) or C1 (0.5 μM) or DMSO (control conditions). Images were collected with a ZEISS 510 Laser Scanning Confocal microscope with a 40× water objective. Slices of 0.5 micron are shown.

Kinase Profiling In Vitro.

The profiling was performed at the MRC (Dundee University), in duplicate, on a set of 121 kinases. The panel is representative of the major different classes of kinases. Compounds were used at the concentration of 1 μM. In vitro, IC50 were determined on recombinant kinases by Reaction biology Corp (USA).

The Multicellular Tumour Spheroid (MTS) Model.

The inventors applied the hanging-drop method to produce H358 spheroids of similar diameters. 1400 cells were suspended on the lid of an agar coated 24-petri dishes containing culture medium. 48 h later, the spheroids were transferred to the culture medium. Spheroid volumes were measured before (Day 0) and during the drug treatment (Day N). H358 spheroids were treated by compounds at the concentration of 0.6 μM. Control H358 spheroids were grown under the same conditions without drug treatment. The size of each spheroid was determined by measuring 2 diameters (d₁ and d₂) using an inverted microscope. The volume was calculated according to the formula: V=4/3π³ where r=½√d₁xd₂. Spheroid growth was calculated by measuring the variations in volume and compared to the initial volume V₀. The growth ratio was defined as (Vd−Vo)/Vo. Data are the mean of at least three determinations on different spheroids.

### REFERENCES

Albanese et al. Mol Cancer Ther. 2010 August; 9(8):2243-54.
Apsel et al, 2008, Nat. Chem. Biol., 4, 291-299
Harel et al. Dev Neurobiol. 2010 April; 70(5):298-303.
Harrison C. Nat Rev Drug Discov. 2010 April; 9(4):270.
Hoang et al., 2009, Cell Cycle, 8, 1-8.
Ippeita et al. TRENDS in Cell Biology 2001: 11 (5) 220-230.
Jetton et al. Mol Microbiol. 2009; 72(2):442-58.
Jian et al., J. Biol. Chem. 2007: 282 (33) 24246-24254
Jian et al. Proc. Natl. Acad. Sci. 2009: 106 (11) 4372-4377.
Liu et al. Nature 2012 Mars 29; 483: 608-612.
Nakano et al. J Neurosci Res. 2008 January; 86(1):48-60.
Peddibhotla et al. Proc Natl Acad Sci USA. 2009; 106(13):5159-64.
Thiele et al. Clin Cancer Res. 2009 Oct. 1; 15(19):5962-7
Watson et al. 2008; 22(6):349-59.

1-23. (canceled)

24. A compound having the formula (I) or (II) 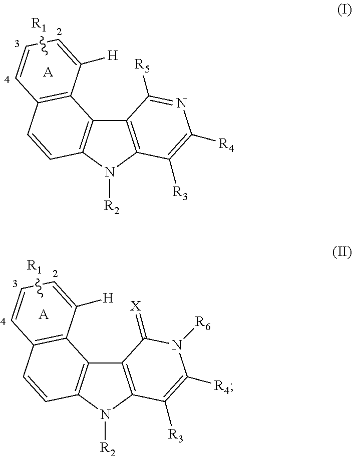
wherein:
the benzo cycle A is mono-substituted by R1 in position 2, 3 or 4;
R1 is a radical —O—(C₂-C₅)alkyl-NRₐR_{b}, wherein (C₂-C₅)alkyl is a linear or branched alkyl, Rₐ and R_{b}, each independently, are selected from the group consisting of hydrogen, (C₁-C₄)alkyl optionally substituted by a radical selected from the group consisting of hydroxyl, —NRR′, OPO(OR)(OR′), and —OC(═O)R, and (C₃-C₆)cycloalkyl; or NRₐR_{b} may be taken together to form a heterocycle selected from the group consisting of aziridine, azetidine, pyrrolidine, pyrrole, piperidine, piperazine, morpholine, and thiomorpholine, the said heterocycle is optionally substituted by a (C₁-C₄)alkyl or hydroxyl radical;
R2 is selected from the group consisting of hydrogen and a (C₁-C₃)alkyl optionally substituted by a radical OH, (C₁-C₃)alkyloxy or —NRR′;
R3 and R4, each independently, are selected from the group consisting of hydrogen, a (C₁-C₃)alkyl and an aryl; or R3 and R4 may be taken together to form a bivalent radical of formula
—(CH₂)ₙ— wherein n is 3, 4 or 5; or
—CH═CH—CH═CH—, optionally substituted by a (C₁-C₃)alkyloxy;
R5, only present in formula (I), is a (C₁-C₄)alkoxy;
R6, only present in formula (II), is selected from the group consisting of hydrogen and a (C₁-C₃)alkyl, optionally substituted by a radical selected from the group consisting of hydroxyl, —NRR′, —OPO(OR)(OR′), and —OC(═O)R;
X, only present in formula (II), is O or S;
wherein R and R′, identical or different, are selected from the group consisting of hydrogen and a (C₁-C₄)alkyl;
or an isomeric form thereof or a pharmaceutically acceptable salt thereof.

25. The compound according to claim 24, wherein the compound of formula (I) or (II) has one or several of the following features:
R1 is a radical —O—(C₂-C₄)alkyl-NRₐR_{b}, wherein (C₂-C₄)alkyl is a linear or branched alkyl, Rₐ and R_{b}, each independently, are selected from the group consisting of hydrogen and a (C₁-C₃)alkyl; or NRₐR_{b} may be taken together to form a heterocycle selected from the group consisting of aziridine, azetidine, pyrrolidine, pyrrole, piperidine, piperazine, morpholine, and thiomorpholine; and/or
R2 is selected from the group consisting of hydrogen, methyl, ethyl, and —(CH₂)ₙ—N[C₁-C₂)alkyl]₂ with n being 2 or 3; and/or
R3 and R4, each independently, are selected from the group consisting of hydrogen, methyl, ethyl and phenyl, or may be taken together to form a bivalent radical of formula —CH═CH—CH═CH—; and/or
in formula (I), R5 is a (C₁-C2)alkoxy; and/or
in formula (II), X is an oxygen; and/or
in formula (II), R6 is selected from the group consisting of hydrogen, methyl, ethyl, —CH₂—OH, —(CH₂)ₙ—N[(C₁-C₂)alkyl]₂ with n being 2 or 3, —(CH₂)—OPO[O(C₁-C₄)alkyl]₂ and —(CH₂)—OC(═O)—(C₁-C₄)alkyl.

26. The compound according to claim 24, wherein the compound of formula (I) or (II) has one or several of the following features:
R1 is a radical —O—(CH₂)ₙ—NRₐR_{b} with n being 2 or 3 or a radical —O—CH₂—(CHCH₃)—CH₂—NRₐR_{b}, wherein Rₐ and R_{b}, each independently, are selected from the group consisting of hydrogen and a (C₁-C₂)alkyl; or NRₐR_{b} may be taken together to form a heterocycle selected from piperidine and morpholine; and/or
R2 is hydrogen; and/or
R3 is selected from the group consisting of hydrogen, methyl and ethyl, and R4 is hydrogen; and/or
in formula (I), R5 is methoxy; and/or
in formula (II), X is an oxygen; and/or
in formula (II), R6 is hydrogen.

27. The compound according to claim 24, wherein
R1 is a radical —O—(CH₂)_{b}—NRₐR_{b} with n being 2 or 3 or a radical —O—CH₂—(CHCH₃)—CH₂—NRₐR_{b}, wherein Rₐ and R_{b}, each independently, are selected from the group consisting of hydrogen and a (C₁-C₂)alkyl; or NRₐR_{b} may be taken together to form a heterocycle selected from piperidine and morpholine;
R2 is hydrogen;
R3 is selected from the group consisting of methyl and ethyl, and R4 is hydrogen; and
either
in formula (I), R5 is methoxy; or
in formula (II), X is an oxygen and R6 is hydrogen.

28. The compound according to claim 24, wherein the compound of formula has the following features:
R1 is a radical —O—(CH₂), —NRₐR_{b} with n being 2 or 3 or a radical —O—CH₂—(CHCH₃)—CH₂—NRₐR_{b}, wherein Rₐ and R_{b}, each independently, are selected from the group consisting of hydrogen and a (C₁-C₂)alkyl; or NRₐR_{b} may be taken together to form a heterocycle selected from the group consisting of piperidine and morpholine;
R2 is hydrogen;
R3 is selected from the group consisting of methyl and ethyl, and R4 is hydrogen; and
X is an oxygen and R6 is hydrogen.

29. The compound according to claim 24, wherein R1 is —O—(CH₂)ₙ—N(CH₃)₂ or —O—(CH₂)ₙ—N(CH₂CH₃)₂ with n being 2 or 3.

30. The compound according to claim 24, wherein R1 is O—(CH₂)₂—N(CH₃)₂.

31. The compound according to claim 24, wherein the compound has the formula (II) with R1 being —O—(CH₂)₂—N(CH₃)₂ (preferably at position 3 of the benzo cycle A), R2, R4 and R6 being hydrogen, X being oxygen and R3 being methyl.

32. The compound according to claim 24, wherein the compound has the formula (II) with R1 being —O—(CH₂)₂—N(CH₃)₂ (preferably at position 3 of the benzo cycle A), R2, R4 and R6 being hydrogen, X being oxygen and R3 being ethyl.

33. The compound according to claim 24, wherein the compound has the formula (II) with R1 being 2-(morpholin-4-yl)ethoxy (preferably at position 3 of the benzo cycle A), R2, R4 and R6 being hydrogen, X being oxygen and R3 being methyl.

34. The compound according to claim 24, wherein the compound has the formula (II) with R1 being 2-(morpholin-4-yl)ethoxy (preferably at position 3 of the benzo cycle A), R2, R4 and R6 being hydrogen, X being oxygen and R3 being ethyl.

35. The compound according to claim 24, wherein the compound has the formula (II) with R1 being 2-(piperidin-1-yl)ethoxy (preferably at position 3 of the benzo cycle A), R2, R4 and R6 being hydrogen, X being oxygen and R3 being methyl.

36. The compound according to claim 24, wherein the compound has the formula (II) with R1 being 2-(piperidin-1-yl)ethoxy (preferably at position 3 of the benzo cycle A), R2, R4 and R6 being hydrogen, X being oxygen and R3 being ethyl.

37. The compound according to claim 24, wherein the compound is selected from the group consisting of:
11-Methoxy-3-(2-N,N-dimethylaminoethoxy)-8-methyl-7H-benzo[e]pyrido(4,3-b)indole;
3-(2-N,N-Dimethylaminoethoxy)-8-methyl-7H,10H-benzo[e]pyrido(4,3-b)indol-11-one (C 14);
3-(3-N,N-Dimethylaminopropoxy)-8-methyl-7H,10H-benzo[e]pyrido(4,3-b)indol-11-one;
3-(2-N,N-Diethylaminoethoxy)-8-methyl-7H,10H-benzo[e]pyrido(4,3-b)indol-11-one;
3-(2-(Morpholin-4-yl)ethoxy)-8-methyl-7H,10H-benzo[e]pyrido(4,3-b)indol-11-one (C07);
3-(2-(Morpholin-4-yl)ethoxy)-8-ethyl-7H,10H-benzo[e]pyrido(4,3-b)indol-11-one;
DL 3-(3-N,N-dimethylamino-2-methylpropoxy)-8-methyl-7H-10H-benzo[e]pyrido(4,3-b)indol-11-one;
3-(3-(Piperidin-1-yl)propoxy)-8-methyl-7H,10H-benzo[e]pyrido(4,3-b)indol-11-one;
3-(2-(Piperidin-1-yl)ethoxy)-8-methyl-7H,10H-benzo[e]pyrido(4,3-b)indol-11-one;
3-(2-(Piperidin-1-yl)ethoxy)-8-ethyl-7H,10H-benzo[e]pyrido(4,3-b)indol-11-one;
3-(2-N,N-dimethylaminoethoxy)-8-ethyl-7H,11H-benzo[e]pyrido(4,3-b)indol-11-one (C48);
and a pharmaceutically acceptable salt thereof, preferably a maleate salt thereof.

38. The compound according to claim 24, wherein the compound is selected from the group consisting of:
3-(2-N,N-Dimethylaminoethoxy)-8-methyl-7H,10H-benzo[e]pyrido(4,3-b)indol-11-one (C14);
3-(2-(Morpholin-4-yl)ethoxy)-8-methyl-7H,10H-benzo[e]pyrido(4,3-b)indol-11-one (C07);
3-(2-(Morpholin-4-yl)ethoxy)-8-ethyl-7H,10H-benzo[e]pyrido(4,3-b)indol-11-one;
3-(2-N,N-dimethylaminoethoxy)-8-ethyl-7H,11H-benzo[e]pyrido(4,3-b)indol-11-one (C48);
3-(2-(Piperidin-1-yl)ethoxy)-8-methyl-7H,10H-benzo[e]pyrido(4,3-b)indol-11-one;
3-(2-(Piperidin-1-yl)ethoxy)-8-ethyl-7H,10H-benzo[e]pyrido(4,3-b)indol-11-one;
and a pharmaceutically acceptable salt thereof, preferably a maleate salt thereof.

39. A pharmaceutical composition comprising a compound according to claim 24 and a pharmaceutically acceptable carrier.

40. The pharmaceutical composition according to claim 39, further comprising an additional antitumoral drug, preferably a DNA-damaging anti-tumoral agent, preferably selected from the group consisting of an inhibitor of topoisomerases I or II, a DNA crosslinker, a DNA alkylating agent, and an anti-metabolic agent.

41. A method for treating cancer, inflammation, pain or parasitic infection in a subject in need thereof, comprising administering an effective amount of a pharmaceutical composition according to claim 39.

42. The method according to claim 41, for treating cancer comprising administering an effective amount of a pharmaceutical composition according to claim 39 in combination with radiotherapy, hyperthermia and/or an antitumoral chemotherapy, preferably a chemotherapy with a DNA-damaging anti-tumoral agent, more preferably a DNA-damaging anti-tumoral agent selected from the group consisting of an inhibitor of topoisomerases I or II, a DNA crosslinker, a DNA alkylating agent, and an anti-metabolic agent.

43. A kit comprising (a) a compound according to claim 24; and (b) an additional antitumoral drug, preferably a DNA-damaging anti-tumoral agent, more preferably an anti-tumoral agent selected from the group consisting of an inhibitor of topoisomerases I or II, a DNA crosslinker, a DNA alkylating agent, and an anti-metabolic agent, as a combined preparation for simultaneous, separate or sequential use, in particular in the treatment of cancer.

44. A method for preparing a compound of formula (I) 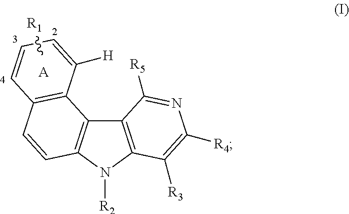
wherein R1, R2, R3, R4 and R5 are as defined in claim 24;
comprising:
a) reacting the reagent Y—(C₂-C₅)alkyl-NRₐR_{b} with the compound 1 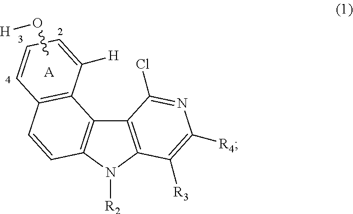
wherein Y is halo, or hydroxy, Rₐ, R_{b}, R2, R3 and R4 are as defined in the present disclosure; and
b) reacting the compound obtained at step a) with alkali (C1-C4)alkoxide, thereby obtaining the compound of formula (I).

45. A method for preparing a compound of formula (II) 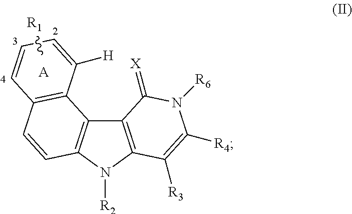
wherein R1, R2, R3, R4, R6 and X are as defined in claim 24;
comprising
a) reacting the reagent Y—(C₂-C₅)alkyl-NRₐR_{b} with the compound 1, 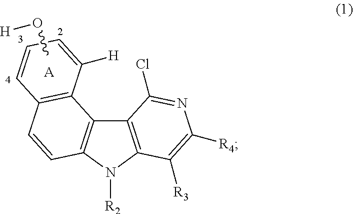
wherein Y is halo or hydroxy, Rₐ, R_{b}, R2, R3, R4 and R6 are as defined in the present disclosure;
and
b) reacting the compound obtained at step a) either with carboxylic acid anhydride or with alkali carboxylate in carboxylic acid, thereby obtaining the compound of formula (II).
